(19) United States
(12) Patent Application Publication
(10) Pub. No.: US 2016/0199499 A1
(43) Pub. Date: Jul. 14, 2016

(54) USES OF PHOSPHOLIPID CONJUGATES OF SYNTHETIC TLR7 AGONISTS

(71) Applicant: THE REGENTS OF THE UNIVERSITY OF CALIFORNIA, Oakland, CA (US)

(72) Inventors: Dennis A. Carson, La Jolla, CA (US); Tomoko Hayashi, San Diego, CA (US); Howard B. Cottam, Escondido, CA (US)

(21) Appl. No.: 14/912,043

(22) PCT Filed: Aug. 14, 2014
(86) PCT No.: PCT/US14/51090

§ 371 Date: Feb. 12, 2016

Related U.S. Application Data
(60) Provisional application No. 61/866,700, filed on Aug. 16, 2013.

(51) Int. Cl. A61K 47/48; A61K 31/522

(52) U.S. Cl. CPC A61K 47/48053; A61K 31/522

(57) ABSTRACT

The invention provides uses for phospholipid conjugates of TLR agonists as enhancers of an innate immune response. Specifically, purine derivatives which are conjugated to a phospholipid or an analog thereof are disclosed as having activity as TLR7 agonists, capable of inducing an innate immune response upon administration in an effective amount to a subject. The phospholipid moiety of the TLR7 agonist contains one or more alkyl ether or ester moieties.

### RELATED PATENT APPLICATIONS

This application claims the benefit of U.S. Provisional Application No. 61/866,700, filed Aug. 16, 2013, entitled “Uses of Phospholipid Conjugates of Synthetic TLR7 Agonists”. The foregoing patent application is incorporated herein by reference in its entirety.

This patent application is related to U.S. patent application Ser. No. 12/027,960, filed on Feb. 7, 2008 and U.S. patent application Ser. No. 13,695,385 filed Apr. 29, 2011. The entire content of the foregoing applications are incorporated herein by reference, including all text, tables and drawings.

### STATEMENT OF GOVERNMENT RIGHTS

The invention described herein was made with government support under Grant Numbers AI077989 and AR062236 awarded by the National Institutes of Health. The United States Government has certain rights in the invention.

### BACKGROUND

A great deal has been learned about the molecular basis of innate recognition of microbial pathogens in the last decade. It is generally accepted that many somatic cells express a range of pattern recognition receptors that detect potential pathogens independently of the adaptive immune system (see Janeway et al., Annu. Rev. Immunol. 20:197 (2002)). These receptors are believed to interact with microbial components termed pathogen associated molecular patterns (PAMPs). Examples of PAMPs include peptidoglycans, lipotechoic acids from gram-positive cell walls, the sugar mannose (which is common in microbial carbohydrates but rare in humans), bacterial DNA, double-stranded RNA from viruses, and glucans from fungal cell walls. PAMPs generally meet certain criteria that include (a) their expression by microbes but not their mammalian hosts, (b) conservation of structure across the wide range of pathogens, and (c) the capacity to stimulate innate immunity. Toll-like Receptors (TLRs) have been found to play a central role in the detection of PAMPs and in the early response to microbial infections (see Underhill et al., Curr. Opin. Immunol. 14:103 (2002)).

Ten mammalian TLRs and a number of their agonists have been identified. For example, guanine and uridine-rich single-stranded RNA has been identified as a natural ligand for TLR7 (Diebold et al., Science 303:1529 (2004)). In addition, several low molecular weight activators of TLR7 have been identified, including imidazoquinolines, and purine-like molecules (Hemmi et al., Nat. Immunol. 3:191 (2002); Lee et al., Proc. Natl. Acad. Sci. USA, 180:6646 (2003); Lee et al., Nat. Cell Biol. 8:1327 (2006)). Among the latter, 9-benzyl-8-hydroxy-2-(2-methoxyethoxy) adenine (“SM”), has been identified as a potent and specific TLR7 agonist. The synthetic immunomodulator R-848 (resiquimod) activates both TLR7 and TLR8. While TLR stimulation initiates a common signaling cascade (involving the adaptor protein MyD88, the transcription factor NF-kB, and pro-inflammatory and effector cytokines), certain cell types tend to produce certain TLRs. For example, TLR7 and TLR9 are found predominantly on the internal faces of endosomes in dendritic cells (DCs) and B lymphocytes (in humans; mouse macrophages express TLR7 and TLR9). TLR8, on the other hand, is found in human blood monocytes (see Hornung et al., J. Immunol., 168:4531 (2002)).

### SUMMARY

Provided herein are methods of using a synthetic TLR7 agonist linked via a stable covalent bond to a phospholipid macromolecule (a conjugate), i.e., the conjugate does not act as a prodrug. The conjugates may include phospholipid macromolecules directly linked to a synthetic TLR7 agonist or linked via a linker to the TLR7 agonist, for instance, linked via an amino group, a carboxy group or a succinamide group. The conjugates are broad-spectrum, transient, and non-toxic synthetic immunostimulatory agents, which are useful for activating the immune system of a mammal, e.g., a human, in vivo by stimulating the activity of TLR7. In particular, the conjugates optimize the immune response while limiting undesirable systemic side effects associated with unconjugated TLR7 agonists.

Thus, in certain aspects are methods of inducing an innate immune response in a mammal, comprising administering to the mammal an effective amount of a compound of formula (I):

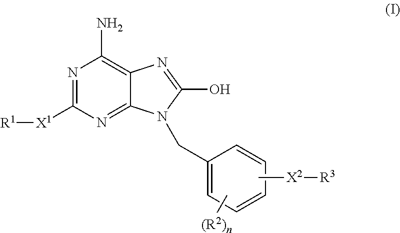

wherein X¹ is —O—, —S—, or —NR^{c}—;

R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;

R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)-(C₁-C₆)alkyl (alkanoyl), substituted —C(O)-(C₁-C₆)alkyl, —C(O)-(C₆-C₁₀)aryl (aroyl), substituted —C(O)-(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

n is 0, 1, 2, 3 or 4;

X² is a bond or a linking group; and

R³ is a phospholipid, or analog thereof comprising one or two alkyl ethers or carboxylic esters of the glyceryl moiety;

or a tautomer thereof;

or a pharmaceutically acceptable salt or solvate thereof.

In some embodiments R³ comprises a group of formula

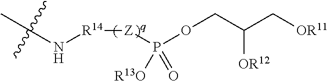

wherein R¹¹ and R¹² are each independently a hydrogen, a C₈-C₂₅ alkyl group or a C₈-C₂₅ acyl group, provided that at least one of R¹¹ and R¹² is an alkyl or an acyl group; R¹³ is a negative charge or a hydrogen, and R¹⁴ is a C₁-C₈ n-alkyl or branched alkyl group which can be substituted or unsubstituted, wherein optionally one of the carbon atoms of the alkyl group is replaced by NH, S, or O; Z is O, S, or NH, and q is 0 or 1; wherein a wavy line indicates a position of bonding, wherein an absolute configuration at the carbon atom bearing OR¹² is R, S, or any mixture thereof. In one embodiment, R¹⁴ is substituted or unsubstituted C₁-C₈ alkyl chain wherein one of the carbons may be substituted with a heteroatom selected from N or S, wherein if the heteroatom is N there is at least on carbon atom between that substituted N and the amine group, e.g., the linker does not include NH—NH. In one embodiment, Z is not O. In one embodiment q=0. In certain embodiments, R¹⁴ is a C₁ alkyl group, in other embodiments R¹⁴ is a C₂ alkyl group. In certain embodiments, R¹⁴ is a branched alkyl group. In some embodiments R¹¹ and R¹² independently are C₈-C₂₅ alkyl. In certain embodiments m is 1.

In some embodiments R¹¹ and R¹² independently are —C(O)-(C₈-C₂₄ alkyl). In certain embodiments R¹¹ and R¹² are each oleoyl groups. In some embodiments the phospholipid of R³ comprises two carboxylic esters and each carboxylic ester includes one, two, three or four sites of unsaturation, epoxidation, hydroxylation, or a combination thereof. In certain embodiments each carboxylic ester of the phospholipid is a C18 carboxylic ester with a site of unsaturation at C9-C10.

In some embodiments X² is a bond, a carbonyl group or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups. In certain embodiments R³ is 1,2-dioleoyl-sn-glycero-3-phospho ethanolamine and X² is a carbonyl moiety (e.g., C(O)).

In certain embodiments X¹ is oxygen.

In some embodiments R¹ is hydrogen, methyl, ethyl, propyl, butyl, hydroxyC₁₋₄alkylene, or C₁₋₄alkoxyC₁₋₄alkylene.

In certain embodiments, each ester of the phospholipid is a C₁₈ ester that is saturated.

In some embodiments, R¹¹ and R¹² are saturated C₈-C₂₅ alkyl groups, providing a glyceryl ether. In some embodiments, R¹¹, R¹², or both, are C₁₈ alkyl groups, which can be saturated or mono-unsaturated.

When q is 0, a direct carbon-phosphorus bond exists between the R¹⁴ group and the phosphorus atom that is also bonded to the glyceryl moiety. This is termed a phosphonate analog of a phospholipid when R¹¹ and R¹² are acyl groups or hydrogen, and is termed a phosphonate analog of a phospholipid glyceryl ether when at least one of R¹¹ and R¹² is an alkyl group.

When q is 1, and Z is 0, the carbon atom of R¹⁴ forms a phosphate ester through an oxygen atom with the phosphorus atom. When R¹¹ and R¹² are acyl groups, this is termed a phospholipid. When at least one of R¹¹ and R¹² is an alkyl group, this is termed a phospholipid glyceryl ether.

When q is 1, and Z is S, the carbon atom of R¹⁴ forms a phosphate thioester through the sulfur atom with the phosphorus atom. When q is 1 and Z is NH, the carbon atom of R¹⁴ forms a phosphoramide through the nitrogen atom to the phosphorus atom.

In some embodiments, q is 0, providing a phosphonate analog of a phospholipid or a phosphonate analog of a phospholipid glyceryl ether. In some embodiments, q is 1, providing a phospholipid (wherein R¹¹ or R¹² or both are acyl groups) or providing a phospholipid glyceryl ether (wherein R¹¹ or R¹² or both are alkyl groups. In some embodiments, one of R¹¹ or R¹² can be a hydrogen. In some embodiments, one of R¹¹ and R¹² can be an acyl group and the other be an alkyl group.

In some embodiments, when Z is O and q is 1, R¹¹ and R¹² are not both acyl groups; in some embodiments when Z is O and q is 1, at least one of R¹¹ and R¹² must be an alkyl group.

When X² is a carbonyl group, it can form an amide bond with the NH that is bonded to R¹⁴ of the phospholipid or phospholipid analog moiety.

In certain embodiments X¹ is oxygen.

In some embodiments R¹ is hydrogen, methyl, ethyl, propyl, butyl, hydroxyC₁₋₄alkylene, or C₁₋₄alkoxyC₁₋₄alkylene.

In certain embodiments X¹ is O, R¹ is C₁₋₄alkoxy-ethyl, n is 0, X² is carbonyl, and R³ is 1,2-dioleoylphosphatidyl ethanolamine (DOPE).

Phosphonate linked phospholipid derivatives (i.e., phosphonate analogs of phospholipids) of TLR7 ligands described above, may improve metabolic stability in that a phosphonate derivative utilizes a C—P (phosphonate), N—P (phosphoramide) or S—P (phosphate thioester) linkage rather than a O—P (phosphate) linkage and is therefore more resistant to phospholipase D cleavage. Replacement of carbonyls of the two acyl chains of the glycerol moiety with alkyl groups may avoid cleavage by phospholipases A1 and A2. Replacement of the C—C double bonds in the acyl chains with C—C single bonds may avoid lipid oxidation that commonly occurs at sites of unsaturation.

Accordingly, in various embodiments, a compound for practice of a method of the invention encompasses structures wherein the 8-hydroxy-9-benzyladenine nucleus is conjugated via the linker X² to various phospholipid analogs, including analogs of phosphatidylalkanolamines, wherein there are 1 to 8 carbon atoms between the NH group bonded to R¹⁴ and group Z, when it is present, or to the phosphorus atom. The glyceryl moiety, bonded via a phosphate ester bond to the phosphorus atom, can bear acyl, alkyl, and hydrogen, provided that R¹¹ and R¹² are not both hydrogen.

In some embodiments the mammal is human.

In some embodiments, the compound is administered in the absence of an antigen or adjuvant, e.g., as a pre-event prophylactic or a post-event prophylactic. In some embodiments, the compound is administered as an adjuvant, e.g., in as (part of) or contemporaneously with a vaccine. In some embodiments, the compound is used in combination with antibiotics, immunotherapies or immunoprophylactics including other compounds that enhance the innate immune response.

In certain embodiments X¹ is O, R¹ is C₁₋₄alkoxy-ethyl, n is 0, X² is carbonyl, and R³ is 1,2-dioleoylphosphatidyl ethanolamine (DOPE).

In some embodiments the mammal is a human. In certain embodiments the human is immunocompromised. In certain embodiments the human is elderly or at least 65 years in age. In certain embodiments the human is a young child or under 5 years of age. In certain embodiments the human is pregnant. In certain embodiments the human is a new born.

In some embodiments the compound is administered in the absence of an antigen or adjuvant.

In some embodiments the compound is administered in a single dose. In some embodiments the compound is administered as multiple equivalent doses or multiple partial doses over a number of days.

In certain embodiments the compound is administered prior to exposure to an infectious agent. In some embodiments the agent is administered 1, 2 or 3 days prior to exposure to an infectious agent.

In certain embodiments the compound is administered after exposure to an infectious agent. In some embodiments the compound is administered immediately after exposure to an infectious agent. In some embodiments the compound is administered within 24 hours or a day after exposure to an infectious agent.

In certain embodiments the compound is administered both prior to exposure to an infectious agent and after exposure to the infectious agent.

In some embodiments administration of a compound is to the respiratory system. In certain embodiments administration is pulmonary administration. In some embodiments administration is intranasal administration. In some embodiments pulmonary administration is intratracheally. In some embodiments, administration is by a route other than the respiratory system. In certain embodiments, administration is to a mucous membrane in the subject.

In certain embodiments an innate immune response is localized to nasal or respiratory tissues. In certain embodiments, an innate immune response is localized to mucosa or a mucous membrane. In certain embodiments, an innate immune response is not localized to nasal or respiratory tissues or mucosa or a mucous membrane.

In some embodiments administration of a compound does not induce detectable off target toxic effects.

In certain embodiments an innate immune response is effective to prevent or inhibit infection by an infectious agent. In certain embodiments an innate immune response is effective to treat an infection by an infectious agent. In some embodiments, the enhanced innate immune response is effective to prevent, inhibit or treat a lethal or sub-lethal dose of the infectious agent. In some embodiments infection by an infectious agent is by inhalation. In certain embodiments infection by an infectious agent is by a route other than inhalation. In some embodiments, an innate immune response involves the mucosal immune system (mucosal immunity). In some embodiments, the mucosal immune system protects a subject's mucous membranes from infection by an infectious agent.

In certain embodiments an infectious agent is bacteria. In a specific embodiment a bacterium is B. anthracis. In certain embodiments an infectious agent is a virus. In a specific embodiment a virus is an influenza virus. In a specific embodiment a virus is an encephalitis virus. In a specific embodiment a virus is a vaccinia virus. In a specific embodiment a virus is West Nile Virus. In some embodiments an infectious agent is a fungus.

Compounds disclosed herein can be used in medical therapy, e.g., for prophylaxis or treatment of microbial infections, such as bacterial infections, viral infections, or fungal infections as well as for the manufacture of a medicament for the treatment of a TLR7 associated condition or symptom in which an augmented immune response is indicated, e.g., in diabetics and those with chronic diseases. For example, the conjugates can be provided to individuals who are more susceptible to infection by infectious agents and for whom the ability to mount an immune response to control an infectious agent is reduced compared to that of comparable heathy individuals. Groups or populations at risk for serious illness or mortality caused by an infectious agent include, but are not limited to, the elderly, the very young, newborn, women who are pregnant and individuals whose immune system is compromised due to illness or as a result of a therapeutic treatment (e.g., radiation therapy, chemotherapy). The conjugates can also be used for biodefense, e.g., against B. anthrax or other potentially lethal infectious agents. The conjugates may optionally be employed with an antigen, e.g., spore, protein, glycoprotein, or membrane, or combinations thereof, of an infectious agent.

In some embodiments compounds disclosed herein are provided in a pharmaceutical composition comprising at least one phospholipid conjugate, or a pharmaceutically acceptable salt thereof. In some embodiments, a pharmaceutical composition comprises nanoparticles formed by combining at least one phospholipid conjugate, or a pharmaceutically acceptable salt thereof, in an aqueous solvent, e.g., PBS, or by combining at least one phospholipid conjugate, or a pharmaceutically acceptable salt thereof, and a preparation of phospholipids, e.g., in an aqueous solvent.

### BRIEF DESCRIPTION OF FIGURES

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIGS. 1A-G show pulmonary administration of phospholipid conjugated TLR7 agonist, 1V270, induces local innate immune activation. C57BL/6 mice (n=5/group) were i.t. treated with 1 nmol 1V270 and BAL were harvested 6, 24, 48, and 72 h after administration. BAL fluids were measured by Luminex beads assay. FIG. 1A shows levels of IL-6 in BAL fluids, FIG. 1B shows levels of MCP-1 in BAL fluids, FIG. 1C shows levels of KC in BAL fluids and FIG. 1D shows IP-10 levels in BAL fluids. Data shown are means±SEM. The data are representative of two independent experiments. FIG. 1E shows *p<0.05 compared to vehicle treated mice by one way ANOVA with Dunnett's post-hoc testing. C57BL/6 mice (n=2-3/group) that were i.n. inoculated with CFSE. Mice were then administered i.t. with 1 nmol 1V270 or vehicle and mediastinal lymph nodes were collected 24 h after treatment. The number of CFSE+CD11c+ cells was identified. Data are means±SD of two independent experiments. FIG. 1F and FIG. 1G show *p<0.05 by Student's t test. Mice (n=4) were i.n. administered with 1, 2, and 4 nmol 1V270 or vehicle control and BAL and sera fluids were collected 24h after the treatment. BAL and sera fluids were measured by Luminex beads assay. *p<0.05 compared to vehicle treated mice by one way ANOVA with Dunnett's post-hoc testing. FIG. 1F shows levels of TNFα in BAL and sera fluids and FIG. 1G shows levels of IL-6 in BAL and sera fluids.

FIG. 2 shows that pulmonary administration of phospholipid conjugated TLR7 agonist, 1V270, does not increase the B cell population. Mice were administered 1 nmol 1V270. Mediastinal, cervical, mesentric and inguinal lymph nodes were harvested 7 days post treatment. B cells were identified as a B220+population using fa low cytometric assay. *: p<0.05 and NS; not significant compared to vehicle treated mice by unpaired Student t test.

FIGS. 3A-B show pulmonary phospholipid conjugated TLR7 agonist, 1V270, treatment induces minimum cell infiltration into lung parenchyma and has negligible adverse effects. FIG. 3A shows C57BL/6 mice (n=5) that were i.t. administered with 1 nmol 1V270. Lungs were harvested 6 and 24 h post treatment. Fixed sections were stained with H&E (Original magnification×200). Scale bar: 200 μm. The sections are representative of 5 mice in a group. FIG. 3B shows mice that were i.t. treated with 1 nmol 1V270 or vehicle. FIG. 3C shows wild type or TIr7−/− mice (n=3-4/group) that were i.t. administered with 75 nmol 1V270. Body weights were monitored daily. *p<0.05 compared to vehicle treated mice by one way ANOVA with Dunnett's post-hoc testing.

FIGS. 4A-G show that pulmonary administration of phospholipid conjugated TLR7 agonist, 1V270, promotes neutrophil infiltration in BAL in a TLR7/MyD88 dependent manner. C57BL/6 mice (n=5/group) were i.t. administered with 1 nmol 1V270 and BAL were harvested 6, 24, 48, and 72 h later. FIG. 4A shows the total cell numbers as determined by a Guava cytometer. FIG. 4B shows numbers of neutrophils in BAL fluids identified after Wright Giemsa staining, FIG. 4C shows numbers of mononuclear cells in BAL fluids identified after Wright Giemsa staining. FIG. 4D and FIG. 4E show mice (n=5/group) that were i.t. treated with the indicated doses of 1V270 and BAL were harvested 24 h post treatment. FIG. 4D shows numbers of total cells and FIG. 4E shows numbers of total neutrophils both determined as described above. FIG. 4F and FIG. 4G show wild type, Myd88−/− or TIr7−/− mice (n=5/group) that were i.t. treated with 1 nmol 1V270 and BAL were harvested 24 h post treatment. FIG. 4F shows number of total cells and FIG. 4G shows numbers of total neutrophils both determined as described above. Data shown are means±SEM. The data are representative of two independent experiments. *p<0.05 compared to vehicle treated mice by one way ANOVA with Dunnett's post-hoc testing.

FIGS. 5A-C show pulmonary treatment with phospholipid conjugated TLR7 agonist, 1V270, protects mice from bacterial and viral infections. FIG. 5A shows NJ mice (n=16) that were i.n. treated with 1V270 (1 nmol) or vehicle at 2-week intervals for three times and challenged with heat-activated live Bacillus anthracis spores 4 weeks after the last immunization. Animal survival was monitored daily for up to 30 days. Kaplan-Meier survival curves and log-rank (Mantel-Cox) tests were performed to determine significance. The data were pooled from two independent experiments. FIG. 5B shows 1V270 (1 nmol/mouse) that were administered i.n. to BALB/c mice (N=20 in placebo group; N=10 in 1V270 group) once a day on days −3 and −1 relative to virus exposure. Mice were challenged s.c. with Venezuelan equine encephalitis virus (Trinidad Donkey, NR-332) on day 0. FIG. 5C shows 1V270 (1 nmol/mouse) or placebo were administered i.n. to BALB/c mice (n=20 in placebo group; n=10 in other groups) once a day on days −3 and −1 prior to virus exposure. Mice were challenged i.n. with an influenza A/California/04/2009 (H1N1) virus on day 0. *, **, and ***: p<0.01, p<0.0005 and p<0.0001, respectively, compared to vehicle treated group by Log-rank (Mantel-Cox) test.

FIGS. 6A-B show survival (FIG. 6A) and weight change (FIG. 6B) for C57BL/6 mice that received intranasal instillation of TMX-201(1V-270) immune modulator and were subsequently infected s.c. with West Nile Virus (WNV).

FIGS. 7A-B show mortality (FIG. 7A) and body weight (FIG. 7B) of mice receiving treatment with 1V270 before and/or during a vaccinia (IHD strain) virus infection. Error bars in FIG. 7B represent ±SD. ***P<0.001, compared to placebo.

FIGS. 8A-C show body weight (graphed by times of treatment initiation) of mice receiving treatment with 1V270 during a vaccinia (IHD strain) virus infection. FIG. 8A represents 1V270 treatments given on days −3 and −1 relative to virus exposure. FIG. 8B represents 1V270 treatments given on days −1 and +1. FIG. 8C represents 1V270 treatments given on days +1 and +3. Error bars represent ±SD.

FIGS. 9A-D illustrate phospholipid based structures with increased metabolic stability. A) Schematic of a more stable derivative of IV270. B) Variables in the linker, phosphate (e.g., phosphonate, phosphoramide, and thiophosphate embodiments) and glyceryl substituents (i.e., replacing acyl groups with alkyl groups to provide a glyceryl ether) in the compounds with enhanced metabolic stability. C) Exemplary synthesis for a phospholipid based structure with increased metabolic stability. 1V-209 is reacted with an appropriate aminophosphonate in the presence of a coupling agent such as HATU. D) Amino phosphonate precursor preparation. The aminophosphonate can be prepared as shown with variable chain lengths of the phosphonate linkage.

### DETAILED DESCRIPTION

### Definitions

A composition is comprised of “substantially all” of a particular compound, or a particular form a compound (e.g., an isomer) when a composition comprises at least about 90%, and at least about 95%, 99%, and 99.9%, of the particular composition on a weight basis. A composition comprises a “mixture” of compounds, or forms of the same compound, when each compound (e.g., isomer) represents at least about 10% of the composition on a weight basis. A TLR7 agonist, or a conjugate thereof, can be prepared as an acid salt or as a base salt, as well as in free acid or free base forms. In solution, certain of the compounds may exist as zwitterions, wherein counter ions are provided by the solvent molecules themselves, or from other ions dissolved or suspended in the solvent.

As used herein, the term “isolated” refers to in vitro preparation, isolation and/or purification of a nucleic acid molecule, a peptide or protein, or other molecule so that it is not associated with in vivo substances or is present in a form that is different than is found in nature. Thus, the term “isolated” when used in relation to a nucleic acid, as in “isolated oligonucleotide” or “isolated polynucleotide” refers to a nucleic acid sequence that is identified and separated from at least one contaminant with which it is ordinarily associated in its source. An isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. Hence, with respect to an “isolated nucleic acid molecule”, which includes a polynucleotide of genomic, cDNA, or synthetic origin or some combination thereof, the “isolated nucleic acid molecule” (1) is not associated with all or a portion of a polynucleotide in which the “isolated nucleic acid molecule” is found in nature, (2) is operably linked to a polynucleotide which it is not linked to in nature, or (3) does not occur in nature as part of a larger sequence. The isolated nucleic acid molecule may be present in single-stranded or double-stranded form. When a nucleic acid molecule is to be utilized to express a protein, the nucleic acid contains at a minimum, the sense or coding strand (i.e., the nucleic acid may be single-stranded), but may contain both the sense and anti-sense strands (i.e., the nucleic acid may be double-stranded).

The term “amino acid” as used herein, comprises the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, -methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆) alkyl, phenyl or benzyl ester or amide; or as an -methylbenzyl amide). Other suitable amino and carboxy protecting groups are known to those skilled in the art (see for example, T. W. Greene, Protecting Groups In Organic Synthesis; Wiley: New York, 1981, and references cited therein). For instance, an amino acid can be linked to the remainder of a compound of formula I through the carboxy terminus, the amino terminus, or through any other convenient point of attachment, such as, for example, through the sulfur of cysteine.

The term “toll-like receptor agonist” (TLR agonist) refers to a molecule that binds to a TLR. Synthetic TLR agonists are chemical compounds that are designed to bind to a TLR and activate the receptor.

The term “nucleic acid” as used herein, refers to DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and fluxionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 7-position purine modifications, 8-position purine modifications, 9-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a BHQ, a fluorophore or another moiety.

A “phospholipid” or analog thereof as the term is used herein refers to a glycerol mono- or diester or diether bearing a phosphate group bonded to a glycerol hydroxyl group with an alkanolamine group being bonded as an ester to the phosphate group, of the general formula

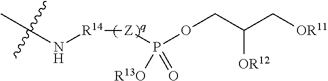

wherein R¹¹ and R¹² are each independently a hydrogen, a C₈-C₂₅ alkyl group or a C₈-C₂₅ acyl group, provided that at least one of R¹¹ and R¹² is an alkyl or an acyl group; R¹³ is a negative charge or a hydrogen, and R¹⁴ is a C₁-C₈ n-alkyl or branched alkyl group which can be substituted or unsubstituted, wherein optionally one of the carbon atoms of the R¹⁴ alkyl group may be replaced by NH, S, or 0; Z is O, S, or NH, and q is 0 or 1; wherein a wavy line indicates a position of bonding, wherein an absolute configuration at the carbon atom bearing OR¹² is R, S, or any mixture thereof.

R¹³ is a negative charge or a hydrogen, depending upon pH. When R₁₃ is a negative charge, a suitable counterion, such as a sodium ion, can be present. In one embodiment, R¹⁴ is substituted or unsubstituted C₁-C₇ alkyl chain wherein one of the carbons may be substituted with a heteroatom selected from N or S. For example, the alkanolamine moiety can be an ethanolamine moiety, such that m=1. It is also understood that the NH group can be protonated and positively charged, or unprotonated and neutral, depending upon pH. For example, the phospholipid can exist as a zwitterion with a negatively charged phosphate oxy anion and a positively charged protonated nitrogen atom. The carbon atom bearing OR¹² is a chiral carbon atom, so the molecule can exist as an R isomer, an S isomer, or any mixture thereof. When there are equal amounts of R and S isomers in a sample of the compound of formula (I), the sample is referred to as a “racemate.” For example in the commercially available product 1,2-dioleoyl-sn-glycero-3-phosphoethanolamine, as used in Example I below, the R³ group is of the chiral structure

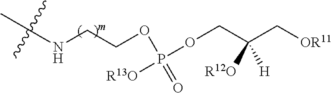

which is of the R absolute configuration (where m is absent or is a C₁-C₈ n-alkyl or branched alkyl group which can be substituted or unsubstituted, wherein optionally one of the carbon atoms of the R¹⁴ alkyl group may be replaced by NH or S but optionally does not form a NH—NH group with the amine).

A phospholipid can be either a free molecule, or covalently linked to another group for example as shown

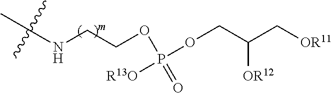

wherein a wavy line indicates a point of bonding (where m is absent or is a C₁-C₈ n-alkyl or branched alkyl group which can be substituted or unsubstituted, wherein optionally one of the carbon atoms of the R¹⁴ alkyl group may be replaced by NH or S but does not form a NH-NH group with the amine).

Accordingly, when a substituent group, such as R³ of the compound of formula (I) herein, is stated to be a phospholipid or analog thereof what is meant that a phospholipid or phospholipid analog group is bonded as specified by the structure to another group, such as to an N-benzyl heterocyclic ring system as disclosed herein. The point of attachment of the phospholipid group can be at any chemically feasible position unless specified otherwise, such as by a structural depiction. For example, in the phospholipid structure shown above, the point of attachment to another chemical moiety can be via the ethanolamine nitrogen atom, for example as an amide group by bonding to a carbonyl group of the other chemical moiety, for example

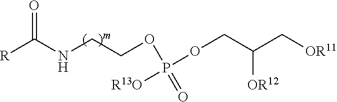

wherein R represents the other chemical moiety to which the phospholipid is bonded. In this bonded, amide derivative, the R¹³ group can be a proton or can be a negative charge associated with a counterion, such as a sodium ion. The acylated nitrogen atom of the alkanolamine group is no longer a basic amine, but a neutral amide, and as such is not protonated at physiological pH.

An “acyl” group as the term is used herein refers to an organic structure bearing a carbonyl group through which the structure is bonded, e.g., to glycerol hydroxyl groups of a phospholipid, forming a “carboxylic ester” group. Examples of acyl groups include fatty acid groups such as oleoyl groups, that thus form fatty (e.g., oleoyl) esters with the glycerol hydroxyl groups. Accordingly, when R¹¹ or R¹², but not both, are acyl groups, the phospholipid shown above is a mono-carboxylic ester, and when both R¹¹ and R¹² are acyl groups, the phospholipid shown above is a di-carboxylic ester.

An “alkyl” group includes straight or branched C₈₋₂₄ alkyl groups which may be substituted. An alkyl group, when bonded to the glyceryl moiety, forms a glyceryl ether. In various embodiments, the compound of formula (I) can be a glyceryl mono- or di-ester. When the compound is a mono-ester, one of R¹¹ and R¹² is an acyl and the other is hydrogen. In other embodiments, the compound of formula (I) can be a glyceryl mono- or di-ether. When the compound is a mono-ether, one of R¹¹ and R¹² is an alkyl and the other is hydrogen. In other embodiments, the compound of formula (I) can be a mixed glyceryl ester-ether, where one of R¹¹ and R¹² is an acyl and the other is an alkyl group.

It is to be understood that a compound of the formula (I) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that any tautomeric form is encompassed, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

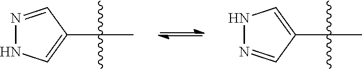 [SMILES: *c1cn[nH]c1 ; *c1cn[nH]c1]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

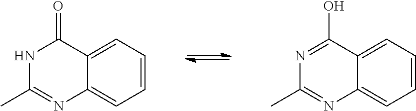

is an example of tautomerism. Accordingly, a structure depicted herein as one tautomer is intended to also include the other tautomer.

### Optical Isomerism

It will be understood that when compounds described herein contain one or more chiral centers, the compounds may exist in, and may be isolated as pure enantiomeric or diastereomeric forms or as racemic mixtures. Iincluded is any possible enantiomers, diastereomers, racemates or mixtures thereof of the compounds described herein.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

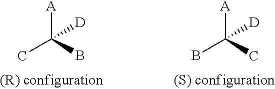

Diastereomers as well as their racemic and resolved, diastereomerically and enantiomerically pure forms and salts thereof are meant to be encompassed. Diastereomeric pairs may be resolved by known separation techniques including normal and reverse phase chromatography, and crystallization.

“Isolated optical isomer” means a compound which has been substantially purified from the corresponding optical isomer(s) of the same formula. In some embodiments, the isolated isomer is at least about 80%, e.g., at least 90%, 98% or 99% pure, by weight. Isolated optical isomers may be purified from racemic mixtures by well-known chiral separation techniques. According to one such method, a racemic mixture of a compound, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

As used herein, “pharmaceutically acceptable salts” refer to derivatives of the disclosed compounds where the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, behenic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds described herein can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile may be employed. Lists of suitable salts are found in Remington's Pharmaceutical Sciences 17th ed., Mack Publishing Company, Easton, Pa., p. 1418 (1985), the disclosure of which is hereby incorporated by reference.

The compounds of the formulas described herein can be solvates, and in some embodiments, hydrates. The term “solvate” refers to a solid compound that has one or more solvent molecules associated with its solid structure. Solvates can form when a compound is crystallized from a solvent. A solvate forms when one or more solvent molecules become an integral part of the solid crystalline matrix upon solidification. The compounds of the formulas described herein can be solvates, for example, ethanol solvates. Another type of a solvate is a hydrate. A “hydrate” likewise refers to a solid compound that has one or more water molecules intimately associated with its solid or crystalline structure at the molecular level. Hydrates can form when a compound is solidified or crystallized in water, where one or more water molecules become an integral part of the solid crystalline matrix.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Het can be heteroaryl, which encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds described herein having a chiral center may exist in and be isolated in optically active and racemic forms. Some compounds may exhibit polymorphism. It is to be understood that any racemic, optically-active, polymorphic, or stereoisomeric form, or mixtures thereof, of a compound described herein, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine agonist activity using the standard tests described herein, or using other similar tests which are well known in the art. It is also understood by those of skill in the art that the compounds described herein include their various tautomers, which can exist in various states of equilibrium with each other.

The terms “treat” and “treating” as used herein refer to (i) preventing a pathologic condition from occurring (e.g., prophylaxis); (ii) inhibiting the pathologic condition or arresting its development; (iii) relieving the pathologic condition; and/or (iv) ameliorating, alleviating, lessening, and removing one or more symptoms of a condition. A candidate molecule or compound described herein may be in an amount in a formulation or medicament, which is an amount that can lead to a biological effect, or lead to protection from, ameliorating, alleviating, lessening, relieving, diminishing or a disease condition, e.g., infection, for example. These terms also are applicable to reducing a titre of a microorganism (microbe) or infectious agent in a system (e.g., cell, tissue, or subject) infected with a microbe, reducing the rate of microbial propagation, reducing the duration of infection of an infectious agent, delaying or attenuating an infection by an infectious agent, reducing the number of symptoms or an effect of a symptom associated with the microbial infection, and/or removing detectable amounts of the microbe from the system. Examples of symptoms include but are not limited weight loss, fever, malaise, weakness, dehydration, failure or diminished organ or organ system function (e.g., pulmonary function). Examples of microbes include but are not limited to viruses, bacteria and fungi.

The term “therapeutically effective amount” as used herein refers to an amount of a compound, or an amount of a combination of compounds, to treat or prevent a disease or disorder or a microbial infection, or to treat or prevent a symptom of the disease or disorder or microbial infection, in a subject. As used herein, the terms “subject” and “patient” generally refers to an individual who will receive or who has received treatment (e.g., administration of a compound) according to a method described herein.

The term “immunocompromised” as used herein refers to a subject having an immune system or portion thereof that is impaired or destroyed such that the ability to prevent, control, or alleviate infection by an infectious agent or mitigate symptoms of such infection is reduced relative to that of an immune system of a comparable (e.g., sex, age, weight, ethnicity, etc.) healthy individual. The subject may be immunocompromised, for example, due to illness or because of receiving treatment (e.g., radiation therapy, chemotherapy or bone marrow transplantation).

The term “elderly” as used herein refers to a subject that is typically 65 years old or greater. Elderly may in include a subject that is at least 50 years old or at least 55 years old, or at least 60 years old. Elderly as used herein refers to any subject that is more prone to infection by an infectious agent and/or has a reduced capacity to prevent, control or alleviate an infection by an infectious agent due in whole or part to aging.

The term “young child” as used herein refers to a subject that is typically under the age of 5 years.

The term lethal dose” as used herein is meant a dose of infectious agent (e.g., number of infectious units or concentration of infectious agent in air or other medium to which a subject is exposed) that results in an infection that causes death. The lethal dose for human can be extrapolated from data obtained from related species challenged by the infectious agent. Lethal doses are usually expressed as median lethal dose (LD50), the point where 50% of test subjects exposed would die. For example, the median lethal dose for humans for anthrax is approximately 2,500 to 55,000 anthrax spores.

The term “sub-lethal dose” as used herein is meant a dose of an infectious agent that is not lethal but which may result in an infection of a subject who may manifest symptoms caused by the infection.

“Stable compound” and “stable structure” are meant to indicate a compound that is sufficiently robust to survive isolation to a useful degree of purity from a reaction mixture, and formulation into an efficacious therapeutic agent. Only stable compounds are contemplated.

### TLR7 Agonists and Conjugates and Uses Thereof

In various embodiments are provided methods to prevent or inhibit infection by an infectious agent in a mammal. The methods include administering to a mammal in need thereof an effective amount of a composition comprising an amount of a compound of Formula (I):

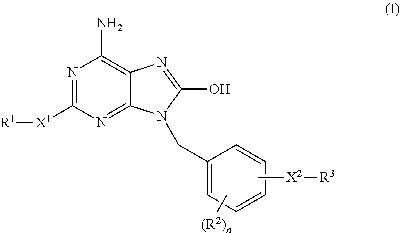

wherein X¹ is —O—, —S—, or —NR^{c}—;

R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;

R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)-(C₁-C₆)alkyl (alkanoyl), substituted —C(O)-(C₁-C₆)alkyl, —C(O)-(C₆-C₁₀)aryl (aroyl), substituted —C(O)-(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

n is 0, 1, 2, 3 or 4;

X² is a bond or a linking group; and

R³ is a phospholipid, or analog thereof comprising one or two alkyl ethers or carboxylic esters of the glyceryl moiety;

or a tautomer thereof;

or a pharmaceutically acceptable salt or solvate thereof.

For example, R³ can comprise a group of formula

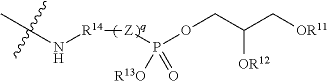

wherein R¹¹ and R¹² are each independently a hydrogen, a C₈-C₂₅ alkyl group or a C₈-C₂₅ acyl group, provided that at least one of R¹¹ and R¹² is an alkyl or an acyl group; R¹³ is a negative charge or a hydrogen, and R¹⁴ is a C₁-C₈ n-alkyl or branched alkyl group which can be substituted or unsubstituted, wherein optionally one of the carbon atoms of the alkyl group is replaced by NH, S, or O; Z is O, S, or NH, and q is 0 or 1;

wherein a wavy line indicates a position of bonding, wherein an absolute configuration at the carbon atom bearing OR¹² is R, S, or any mixture thereof.

An absolute configuration at the carbon atom bearing OR¹² is R, S, or any mixture thereof. In one embodiment, R¹⁴ is substituted or unsubstituted C₁-C₇ alkyl chain wherein one of the carbons may be substituted with a heteroatom selected from N or S.

or

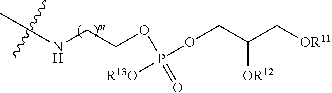

wherein R¹¹ and R¹² are each independently a hydrogen, an alkyl group or an acyl group, R¹³ is a negative charge or a hydrogen, and m is 0 to 8, wherein a wavy line indicates a position of bonding, wherein an absolute configuration at the carbon atom bearing OR¹² is R, S, or any mixture thereof. In one embodiment, m is absent. In one embodiment, m is a C₁-C₈ n-alkyl or branched alkyl group which can be substituted or unsubstituted, wherein optionally one of the carbon atoms of the R¹⁴ alkyl group may be replaced by NH or S.

For example, m can be 1, providing a glycerophosphatidylethanolamine. More specifically, R¹¹ and R¹² can each be oleoyl groups.

In various embodiments, the phospholipid of R³ can comprise two carboxylic esters and each carboxylic ester includes one, two, three or four sites of unsaturation, epoxidation, hydroxylation, or a combination thereof.

In various embodiments, the phospholipid of R³ can comprise two alkyl ethers which may include one, two, three or four sites of unsaturation, epoxidation, hydroxylation, or a combination thereof, or is saturated. In various embodiments, the phospholipid analog of R³ can comprise two glyceryl alkyl ether groups, and the alkyl ethers may be the same or different. More specifically, each ether of the phospholipid analog can be a C17 or C19 saturated alkyl. Alternatively, each ether of the phospholipid analog can be a C18 saturated alkyl.

In various embodiments, the phospholipid of R³ can comprise two carboxylic esters and the carboxylic esters of are the same or different. More specifically, each carboxylic ester of the phospholipid can be a C17 carboxylic ester with a site of unsaturation at C8-C9. Alternatively, each carboxylic ester of the phospholipid can be a C18 carboxylic ester with a site of unsaturation at C9-C10.

In various embodiments, X² can be a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups. The chain can be interspersed with one or more cycloalkyl, aryl, heterocyclyl, or heteroaryl rings.

In various embodiments, X² can be carbonyl (e.g., C(O)), or can be

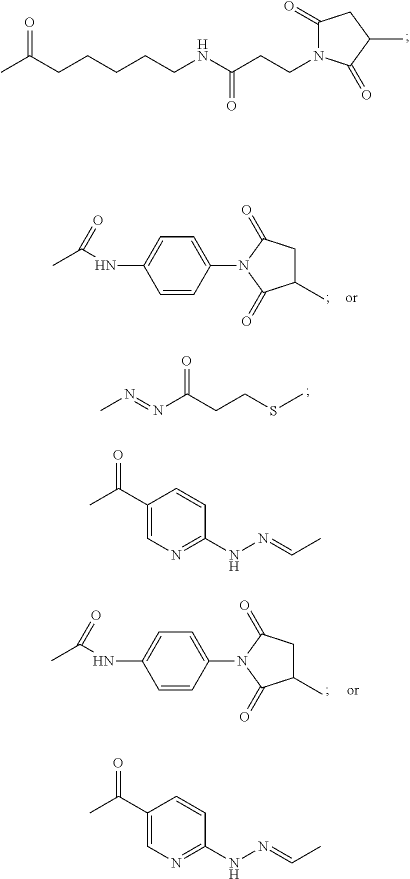

In various embodiments, X² can be

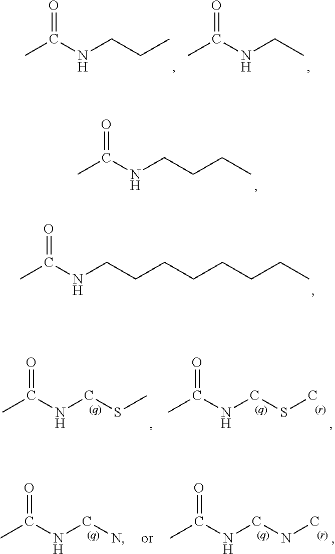

where q=0 to 8 in various embodiments.

In various embodiments, R³ can be dioleoylphosphatidyl ethanolamine (DOPE). In various embodiments R³ is not DOPE.

In various embodiments, R³ can be 1,2-dioleoyl-sn-glycero-3-phospho ethanolamine and X² can be C(O).

In various embodiments, X¹ can be oxygen.

In various embodiments, X¹ can be sulfur, or can be —NR^{c}— where R^{c} is hydrogen, C₁₋₆ alkyl or substituted C₁₋₆ alkyl, where the alkyl substituents are hydroxy, C₃₋₆cycloalkyl, C₁₋₆alkoxy, amino, cyano, or aryl. More specifically, X¹ can be —NH—.

In various embodiments, R¹ and R^{c} taken together can form a heterocyclic ring or a substituted heterocyclic ring. More specifically, R¹ and R^{c} taken together can form a substituted or unsubstituted morpholino, piperidino, pyrrolidino, or piperazino ring.

In various embodiments R¹ can be a C1-C10 alkyl substituted with C1-6 alkoxy.

In various embodiments, R¹ can be hydrogen, C₁₋₄alkyl, or substituted C₁₋₄alkyl. More specifically, R¹ can be hydrogen, methyl, ethyl, propyl, butyl, hydroxyC₁₋₄alkylene, or C₁₋₄alkoxyC₁₋₄alkylene. Even more specifically, R¹ can be hydrogen, methyl, ethyl, methoxyethyl, or ethoxyethyl.

In various embodiments, R² can be absent, or R² can be halogen or C₁₋₄alkyl. More specifically, R² can be chloro, bromo, methyl, or ethyl.

In various embodiments, X¹ can be O, R¹ can be C₁₋₄alkoxy-ethyl, n can be 1, R² can be hydrogen, X² can be carbonyl, and R³ can be 1,2-dioleoylphosphatidyl ethanolamine (DOPE).

In various embodiments, the compound of Formula (I) can be:

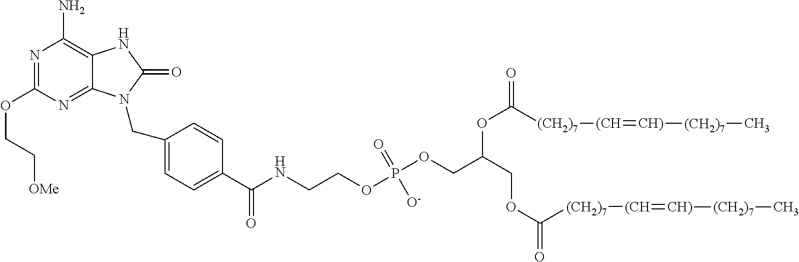

In various embodiments, the compound of formula (I) can be the R-enantiomer of the above structure:

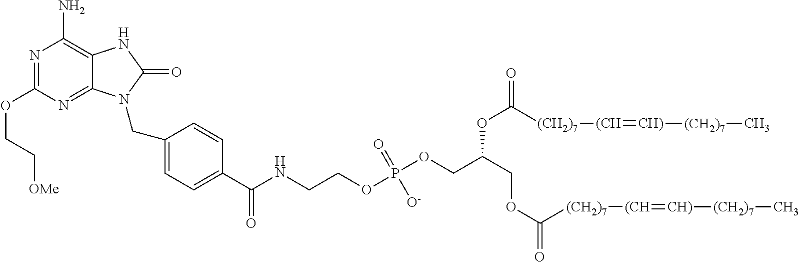

In various embodiments, the compound of formula (I) can be the phospholipid analog conjugate of formula

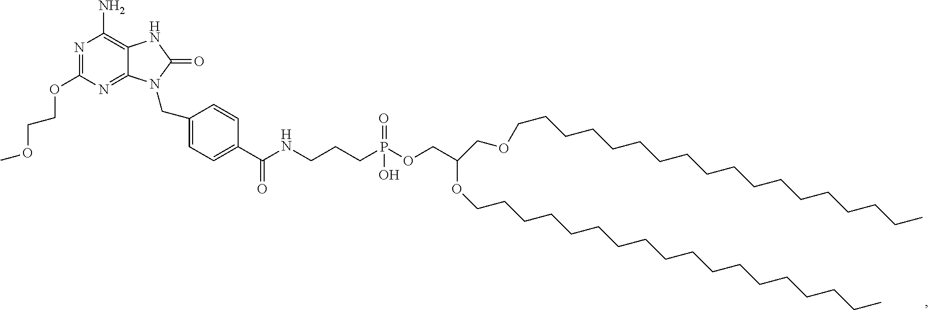 [SMILES: CCCCCCCCCCCCCCCCCCOCC(COP(=O)(O)CCCNC(=O)c1ccc(Cn2c(=O)[nH]c3c(N)nc(OCCOC)nc32)cc1)OCCCCCCCCCCCCCCCCCC]

wherein a phosphonate analog of a phospholipid, having a glyceryl diether group bonded thereto, is conjugated to the benzyladenine moiety via an carboxamide group.

In some embodiments, the composition comprises nanoparticles comprising a compound of formula (I). In various embodiments, a phospholipid conjugate such as 1V270 can be can be incorporated into a nanoparticle such as those described in WO 2010/083337, the disclosure of which is incorporated by reference herein.

As used herein, a nanoparticle has a diameter of about 30 nm to about 600 nm, or a range with any integer between 30 and 600, e.g., about 40 nm to about 250 nm, including about 40 to about 80 or about 100 nm to about 150 nm in diameter. The nanoparticles may be formed by mixing a compound of formula (I), which may spontaneously form nanoparticles, or by mixing a compound of formula (I) with a preparation of lipids, such as phospholipids including but not limited to phosphatidylcholine, phosphatidylserine or cholesterol, thereby forming a nanoliposome. In certain embodiments, a composition forms particles of about 10 nanometers to about 1000 nanometers, and sometimes, a composition forms particles with a mean, average or nominal size of about 100 nanometers to about 400 nanometers.

In various embodiments, a phospholipid conjugate such as 1V270 can be prepared in the form of a nanoparticulate suspension of the phospholipid conjugate in combination with a lipid and/or a phospholipid in an aqueous medium (e.g., a nanoliposome). A nanoliposome is a submicron bilayer lipid vesicle (see Chapter 2 by Mozafari in: Liposomes, Methods in Molecular Biology, vol. 605, V. Weissing (ed.), Humana Press, the disclosure of which is incorporated by reference herein). Nanoliposomes provide more surface area and may increase solubility, bioavailability and targeting.

Optionally, a compound of formula (I), a lipid preparation and a glycol such as propylene glycol are combined.

Lipids are fatty acid derivatives with various head group moieties. Triglycerides are lipids made from three fatty acids and a glycerol molecule (a three-carbon alcohol with a hydroxyl group [OH] on each carbon atom). Mono- and diglycerides are glyceryl mono- and di-esters of fatty acids. Phospholipids are similar to triglycerides except that the first hydroxyl of the glycerol molecule has a polar phosphate-containing group in place of the fatty acid. Phospholipids are amphiphilic, possessing both hydrophilic (water soluble) and hydrophobic (lipid soluble) groups. The head group of a phospholipid is hydrophilic and its fatty acid tail (acyl chain) is hydrophobic. The phosphate moiety of the head group is negatively charged.

In addition to lipid and/or phospholipid molecules, nanoliposomes may contain other molecules such as sterols in their structure. Sterols are important components of most natural membranes, and incorporation of sterols into nanoliposome bilayers can bring about major changes in the properties of these vesicles. The most widely used sterol in the manufacture of the lipid vesicles is cholesterol (Chol). Cholesterol does not by itself form bilayer structures, but it can be incorporated into phospholipid membranes in very high concentrations, for example up to 1:1 or even 2:1 molar ratios of cholesterol to a phospholipid such as phosphatidylcholine (PC) (11). Cholesterol is used in nanoliposome structures in order to increase the stability of the vesicles by modulating the fluidity of the lipid bilayer. In general, cholesterol modulates fluidity of phospholipid membranes by preventing crystallization of the acyl chains of phospholipids and providing steric hindrance to their movement. This contributes to the stability of nanoliposomes and reduces the permeability of the lipid membrane to solutes.

Physicochemical properties of nanoliposomes depend on several factors including pH, ionic strength and temperature. Generally, lipid vesicles show low permeability to the entrapped material. However, at elevated temperatures, they undergo a phase transition that alters their permeability. Phospholipid ingredients of nanoliposomes have an important thermal characteristic, i.e., they can undergo a phase transition (Tc) at temperatures lower than their final melting point (Tm). Also known as gel to liquid crystalline transition temperature, Tc is a temperature at which the lipidic bilayer loses much of its ordered packing while its fluidity increases. Phase transition temperature of phospholipid compounds and lipid bilayers depends on the following parameters: polar head group; acyl chain length; degree of saturation of the hydrocarbon chains; and nature and ionic strength of the suspension medium. In general, Tc is lowered by decreased chain length, by unsaturation of the acyl chains, as well as presence of branched chains and bulky head groups (e.g. cyclopropane rings).

Hydrated phospholipid molecules arrange themselves in the form of bilayer structures via Van-der Waals and hydrophilic/hydrophobic interactions. In this process, the hydrophilic head groups of the phospholipid molecules face the water phase while the hydrophobic region of each of the monolayers face each other in the middle of the membrane. It should be noted that formation of liposomes and nanoliposomes is not a spontaneous process and sufficient energy must be put into the system to overcome an energy barrier. In other words, lipid vesicles are formed when phospholipids such as lecithin are placed in water and consequently form bilayer structures, once adequate amount of energy is supplied. Input of energy (e.g. in the form of sonication, homogenisation, heating, etc.) results in the arrangement of the lipid molecules, in the form of bilayer vesicles, to achieve a thermodynamic equilibrium in the aqueous phase.

For example, a composition comprising a compound such as 1V270 as a mixture with a lipid such as cholesterol or a phospholipid such as phosphatidylcholine can be dispersed into a nanoparticulate form where lipid or phospholipid nanoparticles contain the TLR7 ligand conjugate associated therewith.

For example a nanoparticulate/nanoliposome composition can be prepared using 1V270 and the phophatidylcholine preparation Phosal 50 PG®. 1V270 can be dissolved in Phosal 50 PG (Phospholipid Gmbh, Cologne, Germany) to make a 20× concentrated solution. The Phosal 50 PG-1V270 mixture can be further diluted (1:19) with nanopure water to make a 5% Phosal 50 PG:water suspension. The suspension can be vortexed vigorously and sonicated in a sonicating bath for 10 minutes. The suspension can be further sonicated with a probe sonicater (Branson Sonifier Cell Disrupter 185) at 30% power for a total of 30 seconds at 10 second intervals with 10 seconds rest between so as to not overheat the suspension. Finally, the suspension can be passed through a 100 nm filter with syringe extruder a total of 10 times back and forth. The final nanoparticles can be analyzed with a Malvern Zetasizer to check size distribution. The resulting particles may be referred to as nanoliposomes (a submicron bilayer lipid vesicle) (see Chapter 2 by Mozafari in: Liposomes, Methods in Molecular Biology, vol. 605, V. Weissing (ed.), Humana Press, the disclosure of which is incorporated by reference herein). Nanoliposomes provide more surface area and may increase solubility, bioavailability and targeting.

Nanoparticles are generally stable over time. The particle size of UV-1V270 in PBS is relatively constant with an average of about 110 nm regardless of concentration.

In some embodiments, is provided a prophylactic or therapeutic method for preventing or treating a microbial infection or a pathological condition or symptom in a mammal, such as a human or non-human subject (e.g., bovine, equine, swine, canine, ovine, or feline), where the activity of a TLR7 agonist is implicated and its action is desired. The method includes administering to a mammal in need of such therapy, an effective amount of a conjugate described herein, or a pharmaceutically acceptable salt thereof. Non-limiting examples of pathological conditions or symptoms that are suitable for prophylactic treatment, prevention or inhibition include microbial infections or diseases. In some embodiments, the conjugates described herein can be used to protect a subject from infection by bacteria, viruses, fungi and can be used for biodefense. A conjugate can be used alone or with other therapeutic agents in medical therapy (e.g., for use to prevent or inhibit or bacterial diseases, to prevent or inhibit viral diseases or to prevent or inhibit fungal diseases).

A phospholipid conjugate can be administered to a subject in need thereof to induce, activate, augment, bolster and/or enhance an innate immune response in the subject. In various embodiments, a compound can be administered by a pulmonary route. In various embodiments, a compound can be intranasally administered. In various embodiments, a compound can be administered so as to contact a mucous membrane of a subject.

An innate immune response can be induced by an infectious agent and serves as a first line of defense for rapidly clearing or containing an infection by the infectious agent. Administration of a compound (TLR agonist conjugate) described herein, prior to, during or after exposure of a mammal to an infectious agent can induce an innate immune response which is effective for preventing or inhibiting infection by an infectious agent, when the mammal is subsequently exposed to the infectious agent. A compound can be administered prior to infection by an infectious agent such that the compound acts prophylactically to protect the subject from infection. A compound can be administered after infection by the infectious agent such that the compound augments or enhances an innate immune response induced by the infectious agent.

Characteristics of an innate immune include one or more of activation of cells (e.g., local dendritic cells), induction and release of proinflammatory cytokines (e.g., tumor necrosis factor α (TNF-α), IL-6, IL-12 and type 1 interferon (type 1 IFN), release of chemokines (e.g., monocyte chemoattract protein-1 (MCP-1) and keratinocyte chemoattractants (KC)) and recruitment of cells to the site of the infection e.g., neutrophils.

The TLR agonist conjugates may include a homofunctional TLR agonist, e.g., formed of a TLR7 agonist. The TLR7 agonist can be a 7-thia-8-oxoguanosinyl (TOG) moiety, a 7-deazaguanosinyl (7DG) moiety, a resiquimod moiety, or an imiquimod moiety. In another embodiment, the TLR agonist conjugate may include a heterofunctional TLR agonist polymer. The heterofunctional TLR agonist polymer may include a TLR7 agonist and a TLR3 agonist or a TLR9 agonist, or all three agonists.

In some embodiments, provided are the following conjugates

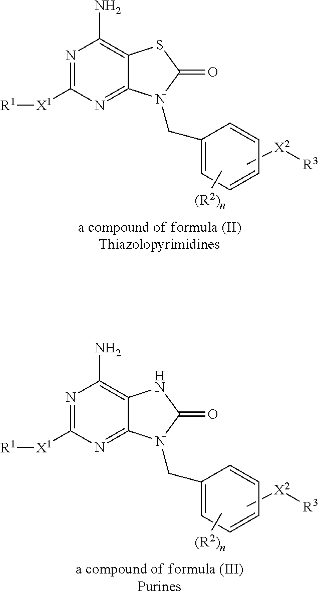

X¹=—O—, —S—, or —NR^{c}—,

wherein R^{c} hydrogen, C₁₋₁₀alkyl, or C₁₋₁₀alkyl substituted by C₃₋₆ cycloalkyl, or R^{c} and R¹ taken together with the nitrogen atom can form a heterocyclic ring or a substituted heterocyclic ring, wherein the substituents are hydroxy, C₁₋₆ alkyl, hydroxy C₁₋₆ alkylene, C₁₋₆ alkoxy, C₁₋₆ alkoxy C₁₋₆ alkylene, or cyano;

wherein R¹ is (C₁-C₁₀)alkyl, substituted (C₁-C₁₀)alkyl, C₆₋₁₀ aryl, or substituted C₆₋₁₀ aryl, C₅₋₉ heterocyclic, substituted C₅₋₉ heterocyc1ic; wherein the substituents on the alkyl, aryl or heterocyclic groups are hydroxy, C₁₋₆ alkyl, hydroxy C₁₋₆ alkylene, C₁₋₆ alkoxy, C₁₋₆ alkoxy C₁₋₆ alkylene, amino, cyano, halogen, or aryl;

each R² is independently —OH, (C₁-C₆)alkyl, substituted (C₁-C₆)alkyl, (C₁-C₆)alkoxy, substituted (C₁-C₆)alkoxy, —C(O)-(C₁-C₆)alkyl (alkanoyl), substituted —C(O)-(C₁-C₆)alkyl, —C(O)-(C₆-C₁₀)aryl (aroyl), substituted —C(O)-(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), —O—C(O)NR^{a}R^{b}, -(C₁-C₆)alkylene-NR^{a}R^{b}, -(C₁-C₆)alkylene-C(O)NR^{a}R^{b}, halo, nitro, or cyano;

wherein each R^{a} and R^{b} is independently hydrogen, (C₁₋₆)alkyl, (C₃-C₈)cycloalky, (C₁₋₆6)alkoxy, halo(C₁₋₆)alkyl, (C₃-C₈)cycloalkyl(C₁₋₆)alkyl, (C₁₋₆)alkanoyl, hydroxy(C₁₋₆)alkyl, aryl, aryl(C₁₋₆)alkyl, aryl, aryl(C₁₋₆)alkyl, Het, Het (C₁₋₆)alkyl, or (C₁₋₆)alkoxycarbony1; wherein X² is a bond or a linking group; wherein R³ is a phospholipid comprising one or two carboxylic esters

wherein n is 0, 1, 2, 3, or 4; or a tautomer thereof; or a pharmaceutically acceptable salt thereof.

In cases where compounds are sufficiently basic or acidic to form acid or base salts, use of the compounds as salts may be appropriate. Examples of acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Alkyl includes straight or branched C₁₋₁₀ alkyl groups, e.g., methyl, ethyl, propyl, butyl, pentyl, isopropyl, isobutyl, 1-methylpropyl, 3-methylbutyl, hexyl, and the like.

Lower alkyl includes straight or branched C₁₋₆ alkyl groups, e.g., methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 1,1-dimethylpropyl, 1,2-dimethylpropyl, 2,2-dimethylpropyl, and the like.

The term “alkylene” refers to a divalent straight or branched hydrocarbon chain (e.g., ethylene: —CH₂—CH₂—).

C₃₋₇ Cycloalkyl includes groups such as, cyclopropyl, cyclopentyl, cyclohexyl, cycloheptyl, and the like, and alkyl-substituted C₃₋₇ cycloalkyl group, e.g., straight or branched C₁₋₆ alkyl group such as methyl, ethyl, propyl, butyl or pentyl, and C₆₋₇ cycloalkyl group such as, cyclopentyl or cyclohexyl, and the like.

Lower alkoxy includes C₁₋₆ alkoxy groups, such as methoxy, ethoxy or propoxy, and the like.

Lower alkanoyl includes C₁₋₆ alkanoyl groups, such as formyl, acetyl, propanoyl, butanoyl, pentanoyl or hexanoyl, and the like.

C₇₋₁₁ aroyl, includes groups such as benzoyl or naphthoyl;

Lower alkoxycarbonyl includes C₂₋₇ alkoxycarbonyl groups, such as methoxycarbonyl, ethoxycarbonyl or propoxycarbonyl, and the like.

Lower alkylamino group means amino group substituted by C₁₋₆ alkyl group, such as, methylamino, ethylamino, propylamino, butylamino, and the like.

Di(lower alkyl)amino group means amino group substituted by the same or different and C₁₋₆ alkyl group (e.g., dimethylamino, diethylamino, ethylmethylamino).

Lower alkylcarbamoyl group means carbamoyl group substituted by C₁₋₆ alkyl group (e.g., methylcarbamoyl, ethylcarbamoyl, propylcarbamoyl, butylcarbamoyl).

Di(lower alkyl)carbamoyl group means carbamoyl group substituted by the same or different and C₁₋₆ alkyl group (e.g., dimethylcarbamoyl, diethylcarbamoyl, ethylmethylcarbamoyl).

Halogen atom means halogen atom such as fluorine atom, chlorine atom, bromine atom or iodine atom.

Aryl refers to a C₆₋₁₀ monocyclic or fused cyclic aryl group, such as phenyl, indenyl, or naphthyl, and the like.

Heterocyclic or heterocycle refers to monocyclic saturated heterocyclic groups, or unsaturated monocyclic or fused heterocyclic group containing at least one heteroatom, e.g., 0-3 nitrogen atoms NR^{c}, 0-1 oxygen atom (—O—), and 0-1 sulfur atom (—S—). Non-limiting examples of saturated monocyclic heterocyclic group includes 5 or 6 membered saturated heterocyclic group, such as tetrahydrofuranyl, pyrrolidinyl, morpholinyl, piperidyl, piperazinyl or pyrazolidinyl. Non-limiting examples of unsaturated monocyclic heterocyclic group includes 5 or 6 membered unsaturated heterocyclic group, such as furyl, pyrrolyl, pyrazolyl, imidazolyl, thiazolyl, thienyl, pyridyl or pyrimidinyl. Non-limiting examples of unsaturated fused heterocyclic groups includes unsaturated bicyclic heterocyclic group, such as indolyl, isoindolyl, quinolyl, benzothizolyl, chromanyl, benzofuranyl, and the like. A Het group can be a saturated heterocyclic group or an unsaturated heterocyclic group, such as a heteroaryl group.

R^{c} and R¹ taken together with the nitrogen atom to which they are attached can form a heterocyclic ring. Non-limiting examples of heterocyclic rings include 5 or 6 membered saturated heterocyclic rings, such as 1-pyrrolidinyl, 4-morpholinyl, 1-piperidyl, 1-piperazinyl or 1-pyrazolidinyl, 5 or 6 membered unsaturated heterocyclic rings such as 1-imidazolyl , and the like.

The alkyl, aryl, heterocyclic groups of R¹ can be optionally substituted with one or more substituents, wherein the substituents are the same or different, and include lower alkyl; cycloalkyl, hydroxyl; hydroxy C₁₋₆ alkylene , such as hydroxymethyl, 2-hydroxyethyl or 3-hydroxypropyl; lower alkoxy; C₁₋₆ alkoxy C₁₋₆ alkyl , such as 2-methoxyethyl, 2-ethoxyethyl or 3-methoxypropyl; amino; alkylamino; dialkyl amino; cyano; nitro; acyl; carboxyl; lower alkoxycarbonyl; halogen; mercapto; C₁₋₆ alkylthio, such as, methylthio, ethylthio, propylthio or butylthio; substituted C₁₋₆ alkylthio, such as methoxyethylthio, methylthioethylthio, hydroxyethylthio or chloroethylthio; aryl; substituted C₆₋₁₀ monocyclic or fused-cyclic aryl, such as 4-hydroxyphenyl, 4-methoxyphenyl, 4-fluorophenyl, 4-chlorophenyl or 3,4-dichlorophenyl; 5-6 membered unsaturated heterocyclic, such as furyl, pyrrolyl, pyrazolyl, imidazolyl, thiazolyl, thienyl, pyridyl or pyrimidinyl; and bicyclic unsaturated heterocyclic, such as indolyl, isoindolyl, quinolyl, benzothiazolyl, chromanyl, benzofuranyl or phthalimino. In certain embodiments, one or more of the above groups can be expressly excluded as a substituent of various other groups of the formulas.

The alkyl, aryl, heterocyclic groups of R² can be optionally substituted with one or more substituents, wherein the substituents are the same or different, and include hydroxyl; C₁₋₆ alkoxy, such as methoxy, ethoxy or propoxy; carboxyl; C₂₋₇ alkoxycarbonyl, such as methoxycarbonyl, ethoxycarbonyl or propoxycarbonyl) and halogen.

The alkyl, aryl, heterocyclic groups of R^{c} can be optionally substituted with one or more substituents, wherein the substituents are the same or different, and include C₃₋₆ cycloalkyl; hydroxyl; C₁₋₆alkoxy; amino; cyano; aryl; substituted aryl, such as 4-hydroxyphenyl, 4-methoxyphenyl, 4-chlorophenyl or 3,4-dichlorophenyl; nitro and halogen.

The heterocyclic ring formed together with R^{c} and R¹ and the nitrogen atom to which they are attached can be optionally substituted with one or more substituents, wherein the substituents are the same or different, and include C₁₋₆ alkyl; hydroxy C₁₋₆ alkylene; C₁₋₆ alkoxy C₁₋₆ alkylene; hydroxyl; C₁₋₆ alkoxy; and cyano. A specific value for X¹ is a sulfur atom, an oxygen atom or —NR^{c}—.

Another specific X¹ is a sulfur atom.

Another specific X¹ is an oxygen atom.

Another specific X¹ is —NR^{c}—.

Another specific X¹ is —NH—.

A specific value for R^{c} is hydrogen, C₁₋₄ alkyl or substituted C₁₋₄ alkyl. A specific value for R¹ and R^{c} taken together is when they form a heterocyclic ring or a substituted heterocyclic ring.

Another specific value for R¹ and R^{c} taken together is substituted or unsubstituted morpholino, piperidino, pyrrolidino, or piperazino ring

A specific value for R¹ is hydrogen, C₁₋₄alkyl, or substituted C₁₋₄alkyl.

Another specific R¹ is 2-hydroxyethyl, 3-hydroxypropyl, 4-hydroxybutyl, 2-aminoethyl, 3-aminopropyl, 4-aminobutyl, methoxymethyl, 2-methoxyethyl, 3-methoxypropyl, ethoxymethyl, 2-ethoxyethyl, methylthiomethyl, 2-methylthioethyl, 3-methylthiopropyl, 2-fluoroethyl, 3-fluoropropyl, 2,2,2-trifluoroethyl, cyanomethyl, 2-cyanoethyl, 3-cyanopropyl, methoxycarbonylmethyl, 2-methoxycarbonylethyl, 3-methoxycarbonylpropyl, benzyl, phenethyl, 4-pyridylmethyl, cyclohexylmethyl, 2-thienylmethyl, 4-methoxyphenylmethyl, 4-hydroxyphenylmethyl, 4-fluorophenylmethyl, or 4-chlorophenylmethyl.

Another specific R¹ is hydrogen, CH₃—, CH₃-CH₂—, CH₃CH₂CH₂—, hydroxyC₁₋₄alkylene, or C₁₋₄alkoxyC₁₋₄alkylene.

Another specific value for R¹ is hydrogen, CH₃—, CH₃—CH₂—, CH₃—O—CH₂CH₂— or CH₃—CH₂—O—CH₂CH₂—.

A specific value for R² is halogen or C₁₋₄alkyl.

Another specific value for R² is chloro, bromo, CH₃—, or CH₃—CH₂—.

Specific substituents for substitution on the alkyl, aryl or heterocyclic groups are hydroxy, C₁₋₆alkyl, hydroxyC₁₋₆alkylene, C₁₋₆alkoxy, C₁₋₆alkoxyC₁₋₆alkylene, C₃₋₆cycloalkyl, amino, cyano, halogen, or aryl.

A specific value for X² is a bond or a chain having up to about 24 atoms; wherein the atoms are selected from the group consisting of carbon, nitrogen, sulfur, non-peroxide oxygen, and phosphorous. Any carbon atom can bear an oxo group, and any sulfur atom can bear one or two oxo groups. The chain can be interspersed with one or more cycloalkyl, aryl, heterocyclyl, or heteroaryl rings.

Another specific value for X² is a bond or a chain having from about 4 to about 12 atoms.

Another specific value for X² is a bond or a chain having from about 6 to about 9 atoms.

Another specific value for X² is a carbonyl (C(O)) group.

Certain non-limiting examples of X² include —(Y)_{y}—, —(Y)_{y}—C(O)N—(Z)_{z}—, —(CH₂)_{y}—C(O)N—(CH₂)_{z}—, —(Y)_{y}—NC(O)—(Z)_{z}—, —(CH₂)_{y}—NC(O)—(CH₂)_{z}—, where each y (subscript) and z (subscript) independently is 0 to 20 and each Y and Z independently is C1-C10 alkyl, substituted C1-C10 alkyl, C1-010 alkoxy, substituted C1-010 alkoxy, C3-C9 cycloalkyl, substituted C3-C9 cycloalkyl, C5-C10 aryl, substituted C5-C10 aryl, C5-C9 heterocyclic, substituted C5-C9 heterocyclic, C1-C6 alkanoyl, Het, Het C1-C6 alkyl, or C1-C6 alkoxycarbonyl, wherein the substituents on the alkyl, cycloalkyl, alkanoyl, alkcoxycarbonyl, Het, aryl or heterocyclic groups are hydroxyl, C1-C10 alkyl, hydroxyl C1-C10 alkylene, C1-C6 alkoxy, C3-C9 cycloalkyl, C5-C9 heterocyclic, C1-6 alkoxy C1-6 alkenyl, amino, cyano, halogen or aryl. In certain embodiments, a linker sometimes is a —C(Y′)(Z′)—C(Y″)(Z″)- linker, where each Y′, Y″, Z′ and Z″ independently is hydrogen C1-C10 alkyl, substituted C1-C10 alkyl, C1-C10 alkoxy, substituted C1-C10 alkoxy, C3-C9 cycloalkyl, substituted C3-C9 cycloalkyl, C5-C10 aryl, substituted C5-C10 aryl, C5-C9 heterocyclic, substituted C5-C9 heterocyclic, C1-C6 alkanoyl, Het, Het C1-C6 alkyl, or C1-C6 alkoxycarbonyl, wherein the substituents on the alkyl, cycloalkyl, alkanoyl, alkcoxycarbonyl, Het, aryl or heterocyclic groups are hydroxyl, C1-C10 alkyl, hydroxyl C1-C10 alkylene, C1-C6 alkoxy, C3-C9 cycloalkyl, C5-C9 heterocyclic, C1-6 alkoxy C1-6 alkenyl, amino, cyano, halogen or aryl.

Another specific value for X² is

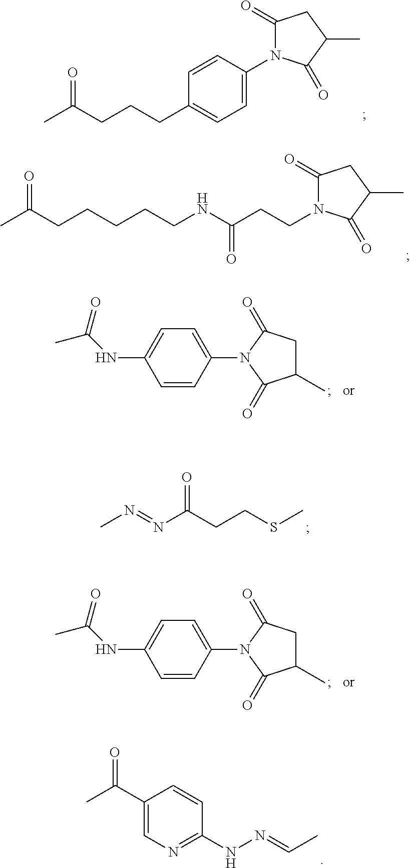

Another specific value for X² is

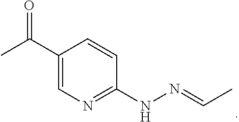

Compositions that include of a TLR7 agonist phospholipid conjugate optionally in combination with other active agents that may or may not be antigens, e.g., ribavirin, mizoribine, and mycophenolate mofetil. Other non-limiting examples are known and are disclosed in U.S. published patent application No. 20050004144.

Synthesis of a phospholipid conjugated ligand, (1V270) (2-(4-{[6-Amino-2-(2-methoxyethoxy)-8-oxo-7H-purin-9(8H)-yl]methyl}benzamido)ethyl 2,3-Bis(oleoyloxy)propyl Phosphate) is described in Chan et al. Bioconjug Chem 2009; 20:1194-1200.

Administration of compositions having conjugates described herein, e.g., administration of a composition having a phospholipid conjugate without another agent, e.g., an antigen, adjuvant or another active agent, administration of a composition having a phospholipid conjugate and another active agent or administration of a composition having a phospholipid conjugate and a composition having another active agent, can be via any of suitable route of administration.

One non-limiting example of a route of administration of a phospholipid conjugate is to the respiratory system. The respiratory system includes the nasal cavity and associated sinuses, the nasopharynx, oropharynx, larynx, trachea, bronchi, bronchioles, respiratory bronchioles, alveolar ducts and alveolar sacs. In specific embodiments the compounds described herein are administered to the lungs or the nasal cavity.

Pulmomary administration can be used for delivery to the lungs and other regions of the respiratory system. Pulmonary administration includes, but is not limited to, aerosol inhalation via nasal (intranasal) or oral routes and intratracheal instillation.

Aerosol inhalation is by any means by which an aerosol can be introduced into the respiratory system, including, but not limited to, pressurized metered dose inhalers, dry power inhalers and nebulisers (e.g., liquid spray and suspension spray) for oral route or any device suitable for intranasal administration.

In addition, in some embodiments, are provided various dosage formulations of the phospholipid conjugate optionally in combination with another active compound for inhalation delivery. For example, formulations may be designed for aerosol use in devices such as metered-dose inhalers, dry powder inhalers and nebulizers.

Intratracheal instillation can be carried out by delivering a solution into the lungs via a device, such as a syringe.

Intranasal administration which can be employed to effect pulmonary administration can be used specifically for administration to the nasal cavity and sinuses. Devises for intranasal administration include, but are not limited to liquid drop devices, spray devices, dry powder devices and aerosol devices. Intranasal administration can also be by nasal gel or insuffulations.

Formulation of the compounds described herein as aerosols (solid or liquid particles), liquids, powders, gels, nanoparticles may be obtained using standard procedures well known in the art.

The phospholipid conjugate optionally in combination with another active compound may also be administered parenterally, for example, intravenously, intra-arterially, intraperitoneally, intrathecally, intraventricularly, intraurethrally, intrasternally, intracranially, intramuscularly, or subcutaneously. Such administration may be as a single bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compounds (a conjugate or other active agent) may be formulated as a sterile solution in water or another suitable solvent or mixture of solvents. The solution may contain other substances such as salts, sugars (particularly glucose or mannitol), to make the solution isotonic with blood, buffering agents such as acetic, critric, and/or phosphoric acids and their sodium salts, and preservatives.

The phospholipid conjugates alone (without antigen or adjuvant) or in combination with other active agents can be formulated as pharmaceutical compositions and administered to a mammalian host, such as a human patient in a variety of forms adapted to the chosen route of administration, e.g., by pulmonary routes, orally or parenterally, by intravenous, intramuscular, topical or subcutaneous routes.

Thus, the present phospholipid conjugates alone or in combination with another active agent, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the conjugate optionally in combination with an active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of conjugate and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the phospholipid conjugate optionally in combination with another active compound may be incorporated into sustained-release preparations and devices.

The phospholipid conjugate optionally in combination with another active compound may also be administered intravenously or intraperitoneally by infusion or injection. Solutions of the phospholipid conjugate optionally in combination with another active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms during storage can be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating compound(s) in the required amount in the appropriate solvent with various of the other ingredients enumerated above, as required, followed by filter sterilization. In the case of sterile powders for the preparation of sterile injectable solutions, one method of preparation includes vacuum drying and the freeze drying techniques, which yield a powder of the active ingredient plus any additional desired ingredient present in the previously sterile-filtered solutions.

For topical administration, the phospholipid conjugate optionally in combination with another active compound may be applied in pure form, e.g., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages can be determined by comparing their in vitro activity, and in vivo activity in animal models. Methods for the extrapolation of effective dosages in mice, and other animals, to humans are known to the art; for example, see U.S. Pat. No. 4,938,949. The ability of a compound to act as a TLR agonist may be determined using pharmacological models which are well known to the art, including the procedures disclosed by Lee et al., Proc. Natl. Acad. Sci. USA 100: 6646 (2003).

Generally, the concentration of the phospholipid conjugate optionally in combination with another active compound in a liquid composition, such as a lotion, will be from about 0.1-25 wt-%, e.g., from about 0.5-10 wt-%. The concentration in a semi-solid or solid composition such as a gel or a powder will be about 0.1-5 wt-%, e.g., about 0.5-2.5 wt-%.

The active ingredient may be administered to achieve peak plasma concentrations of the active compound of from about 0.5 to about 75 μM, e.g., about 1 to 50 μM, such as about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The amount of the phospholipid conjugate optionally in combination with another active compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a suitable dose will be in the range of from about 0.5 to about 100 mg/kg, e.g., from about 10 to about 75 mg/kg of body weight per day, such as 3 to about 50 mg per kilogram body weight of the recipient per day, for instance in the range of 6 to 90 mg/kg/day, e.g., in the range of 15 to 60 mg/kg/day. More than one dose (e.g., 2, 3, 4, 5, 6, 7, 8, 9, 10, 11, 12, 13, 14, 15, 16, 17, 18, 19, 20, 21, 22, 23, 24, 25, 26, 27, or 28, or, for example, 35, 42, 49, 56, 63, or 70) may be determined by a physician or clinician to be required. Doses may be administered before, after, or before and after exposure to the infectious agent as determined by a physician or clinician based on the above discussed factors and other relevant factors. Scheduling of administration of doses (e.g., consecutive days, alternate days, multiple doses in one day) can also be determined by a physician or clinician based on the above discussed factors and other relevant factors.

The duration of treatment with the phospholipid conjugate can be for a predetermined period of time. For example, 1, 2, 3, 4, 5, 6, 7 or more days, one week, two weeks, three weeks, four weeks or more. Alternatively, the duration of treatment with the phospholipid conjugate can be for a period of time until the infectious agent is no longer detectable in the subject or the infectious agent is present at a level that does not result in symptoms or until there is an elimination or reduction in the number or severity of symptoms typically exhibited by a subject infected with a specific infectious agent. The duration of treatment can be determined by a physician or clinician based on the above discussed factors and other relevant factors.

The phospholipid conjugate optionally in combination with another active compound may be conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. The dose, and perhaps the dose frequency, will also vary according to the age, body weight, condition, and response of the individual patient. In general, the total daily dose range for an active agent for the conditions described herein, may be from about 50 mg to about 5000 mg, in single or divided doses. In some embodiments, a daily dose range should be about 100 mg to about 4000 mg, e.g., about 1000-3000 mg, in single or divided doses, e.g., 750 mg every 6 hr of orally administered compound. This can achieve plasma levels of about 500-750 uM, which can be effective to kill cancer cells. In managing the patient, the therapy should be initiated at a lower dose and increased depending on the patient's global response.

In some embodiments the compound is not administered with a solvent or preservative such as DMSO or ethanol, which may have toxic effects, e.g., in humans.

In some embodiments, a single dose of the conjugate may show very potent activity in enhancing an innate immune response. Moreover, because of the low toxicity of the conjugates, in some circumstances higher doses may be administered, e.g., systemically, while under other circumstances lower doses may be administered, e.g., due to localization of the conjugate.

In some embodiments, administration of a therapeutically effective amount of the conjugate to a subject and subsequent challenge by a lethal dose of an infectious agent can achieve a therapeutic response of about 40% or greater. In certain embodiments the therapeutic response can be about 50% or greater, about 60% or greater, about 70% or greater, about 80% or greater, about 90% or greater, about 95% or greater, about 99% or greater or about 100%. The therapeutic endpoint can be based on survival of the subject for a minimum number of days post infection by an infectious agent. The minimum number of days of survival can be specific to a particular infectious agent. In some embodiments, the minimum number of days of survival post infection that can serve as a therapeutic endpoint can be about 14, 13, 12, 11, 10, 9, 8, 7, 6 or 5 days. Other measurements of efficacy of a conjugate that are not based on survival after challenge by a lethal dose of an infectious agent are known to those who practice the art.

In some embodiments, administration of a therapeutically effective amount of the conjugate to a subject who has already been challenged or is subsequently challenge by a sub-lethal dose of an infectious agent achieves a therapeutic response of about 40% or greater. In certain embodiments the therapeutic response can be about 50% or greater, about 60% or greater, about 70% or greater, about 80% or greater, about 90% or greater, about 95% or greater, about 99% or greater or about 100%. The therapeutic response can be based on the elimination of or reduction in the number or severity of symptoms typically exhibited by a subject infected with the specific infectious agent.

In some embodiments, an innate immune response can be localized to the respiratory tract, i.e., nasal or respiratory tissues by delivery of a phospholipid TLR7 agonist conjugate to the respiratory system (e.g., by pulmonary administration). For example, localization of an innate immune response can be evidenced by an increase of innnate cytokines and chemokines in the bronchial alveolar lavage (BAL fluids) and not in serum. Localization of an innate immune response can also be evidenced by an increase in the total number of cells in BAL fluids, especially in the neutrophil population. Localization of an innate immune response does not necessarily preclude the manifestation of a systemic immune response (e.g., circulating T cells). Localization can be evidenced by the display of at least one characteristic of localized immune response in nasal or respiratory tissues (e.g., localized cytokines and/or chemokines or localized increase in cells).

In some embodiments, an innate immune response can be localized to other areas in the subject besides the respiratory tract. In some embodiments these areas have mucous membranes, and include but are not limited to the mouth, eyes, intestines, stomach, urethra, ears, genital areas, endometrium, the birth canal and the anus. Localization to a mucous membrane can be evidenced by a localized increase in cytokines and/or chemokines or localized increase in cells)

In some embodiments, a compound described herein does not induce detectable off target adverse effects. Such adverse effects include, but are not limited to systemic cytokine release (e.g., cytokine syndrome), B cell proliferation in lymphoid organs distal to the site of administration of the compound, weight loss (anorexic behavior) and pulmonary edema or interstitial inflammation, which can be evaluated for example by histological changes in the lung such as cell infiltration into lung parenchyma.

Infection can be by inhalation of an airborne infectious agent, e.g., droplets (from an infected subject coughs or sneezes), viral droplet nucleic transmission (for example by an infected person releasing viruses by talking sneezing, coughing and breathing) or in or on airborne particles (e.g., dust). Infections agents may be intentionally be aerosolized for purposes of biological warfare.

Infection can be by a route other than inhalation such as direct (e.g., by touching an infected subject) or indirect contact (e.g., through a break in the skin), or contacting a contaminated surface, by another organism such as a vector (e.g., an insect) or an intermediate host (e.g. tapeworm). In some embodiments, the infectious agent is not inhaled, however subsequent to infection the infectious agent resides, disperses, proliferates or causes pathological effects in the respiratory system. In some embodiments, the infectious agent is not inhaled and the respiratory system is not utilized by the infectious agent. In some embodiments, the infection is via a mucous membrane at a location in the subject that may not be part of the respiratory system.

Compounds described herein, when administered to the respiratory system, are effective for the prevention or inhibition of airborne infectious agents such as bacteria, viruses and fungi that directly enter the respiratory system by inhalation. In some embodiments, the microbe does not directly enter the respiratory system by inhalation, but enters the subject by other routes instead and accesses nasal or respiratory tissues pursuant to its mechanism of causing disease or pathology. For example, Venezuelan equinine encephalitis (VEE) infection is mediated by a mosquito, but the virus replicates in the nasal epithelium and enters the central nervous system through sensory neurons in the nasal olfactory neuroepithium.

A microbial infection may be caused by a bacterium. Bacteria include, but are not limited to, Neisseria meningitides, Klebsiella pneumonia, Pseudomonas aeruginosa, Pseudomonas mallei, Acinetobacter, Moraxella catarrhalis, Moraxella lacunata, Alkaligenes, Cardiobacterium, Haemophilus influenza, Haemophilis parainfluenzae, Bordetella pertussis, Francisella tularensis, Legionella pneumophila, Chlamydia psittaci, Chlamydia pneumonia, Mycobacterium tuberculosis, Mycobacterium kansaii, Mycobacterium avium-tracell, Nocardia asteroids, Bacillus antracis, Staphlococcus aureus, Streptococcus pyogenes, Streptococcus pneumonia, Corynebacteria diphtheria and Mycoplasma pneumonia. Anthrax infection may be caused by anthrax spores.

A microbial infection may be caused by a virus. Viruses include, but are not limited to, Orthomyxoviridae-Influenza, Arenavirus-Junin, Arenavirus-Machupe, Arenavirus-Lassa, Filovirus-Marburg, Filovirus-Ebola, Hantaviruses, Picornoviridae-Rhinoviruses, Picornoviridae-Echovirus, Coronaviruses, Paramyxovirus, Morbillivirus, Respiratory Synctial Virus, Togavirus, Coxsackievirus, Parvovirus B19, Parainfluenza, Adenoviruses, Reoviruses, Poxvirus-Variola, Poxvirus-Vaccinia, Varicella-zoster, Viral Equimine Encephalitis viruses including Venezuelan equinine encephalitis (VEE), herpes viruses (e.g., Herpes Simplex Virus (HSV-1, HSV-2)) and papilloma viruses including Human Papilloma Viruses (HPV). As the proposed mode of action of the compound is as an immunomodulatory agent and not a typical antiviral agent it may be effective against a number of different viruses.

In certain embodiments the virus is an Influenza virus. A challenge to protecting against infection by Influenza viruses is caused by the high rate of mutation of the virus. Compounds described herein may be especially useful for Influenza viruses as they should not be as vulnerable to the effects of mutations. Protection from influenza virus infection by pulmonary administration of a lipid conjugate may be as high as about 100%.

In certain embodiments the virus is a vaccinia virus.

In certain embodiments the virus is West Nile Virus.

A microbial infection may be caused by a fungus. Fungi include, but are not limited to, Aspergillus spp., Absidia corymbifera, Rhizopus stolonifer, Mucor plumbeus, Cryptococcus neoformans, Histoplasma capsulatum, Blastomyces dermatitidis, Coccidioides immitis, Penicillium spp., Micromonospora faeni, Thermoactinomyces vulgaris, Alternaria alternate, Cladosporium spp. Helminthosporium and Stachybotrys spp.

The invention will be further described by the following non-limiting examples.

### EXAMPLES

The examples set forth below illustrate certain embodiments and do not limit the technology.

Pathogen associated molecules are recognized as danger signals by pattern recognition receptors (PRRs) on innate immune cells that initiate host defense reactions. Among PRRs, Toll like receptors (TLRs) play essential roles in the protective responses against infectious diseases. Virus particles activate the innate immune system via nucleotide receptors, such as TLR3, TLR7, TLR8, or TLR9 [1]. Bacterial infection initiates a broad range of TLR activation, including TLR2, TLR4, TLR5, and TLR9 [2]. Triggering of TLRs initiates protective immunity through activation of signaling pathways that induce a wide variety of anti-microbial host responses [3].

The innate immune response of the respiratory tract is the first line of defense against aerosolized pathogens and may profoundly affect the disease manifestations and outcomes of many viral, bacterial and fungal infections. Failure to develop an early, robust innate immune response may faster microbial colonization and infection in the airway and the lung parenchyma. Prophylactic administration of ligands for TLR2, TLR3, TLR4, and TLR9 has been reported to reduce the severity of various pulmonary infections [4-15]. However, excess TLR activation can also induce severe local and systemic inflammatory reactions. Such safety concerns have impeded the clinical development of TLR ligands as immune protectants [16].

Previously it was reported that covalent conjugation of a modified adenine-based TLR7 agonist to mouse serum albumin (MSA) enhanced its ability to stimulate innate immune responses while reducing drug induced systemic cytokine release [17]. Mice pretreated with the TLR7 ligand-MSA conjugate, delivered via the pulmonary route, and then challenged with Bacillus anthracis spores or H1N1 influenza A virus showed a significant delay in mortality [18]. However, modified proteins may be immunogenic and are difficult to manufacture and to store. The lung is normally bathed in various phospholipids [19]. Therefore, we synthesized 1V270 consisting of a TLR7 agonist conjugated to a physiologic C-16 phospholipid [17]. When 1V270 was used as an adjuvant in a standard vaccination study, potent both Th1 and Th2 antigen-specific immune responses were induced, without the induction of local and systemic inflammation [17]. It was demonstrated that pulmonary administration of 1V270 activates local immune responses without causing lung damage. This phospholipid modified TLR7 ligand activated local dendritic cells and induced with resultant cytokine release into the bronchial alveolar lavage (BAL) fluids. In contrast, pulmonary administration of 1V270 did not cause systemic cytokine release, weight loss, or B cell mitogenesis in the distant lymphoid organs. These results suggest that that 1V270 is a potent inducer of innate immune responses in the lung with appropriate safety profile. This drug may therefore be useful for protection against infection by aerosolized viral and bacterial infection.

### Example 1

### Materials

### Animals

Female C57BL/6, A/J and BALB/c mice were purchased from The Jackson Laboratory (Bar Harbor, Mass.) and Charles River Laboratory (Wilmington, Mass.), respectively. TLR7 and MyD88 deficient mice were a gift from Dr. S. Akira (Osaka University, Osaka, Japan) and bred onto the C57BL/6 background at University of California, San Diego (UCSD). This study was carried out in strict accordance with the recommendations in the Guide for the Care and Use of Laboratory Animals of the National Institutes of Health. All procedures used in this study were approved by the Institutional Animal Care and Use Committees of UCSD and Utah State University.

### Reagents

Phosphate buffered saline (PBS, pH 7.4), RPMI 1640 medium (Life Technologies, Grand Island, N.Y.), DMEM (Life Technologies) were supplemented with 10% fetal bovine serum (FBS, Sigma St Louis) and penicillin/streptomycin (Sigma). Phospholipid conjugated TLR7 ligand, 1V270, was synthesized in as previously described [17]. 1V270 was dissolved in DMSO as a 10 mM stock solution and kept at −20° C. until use. Endotoxin contamination was ruled out by the finding of similar potencies of this compound in TIr4−/− and wild type bone marrow cells [17].

### Methods

### Intranasal (i.n.) and Intratracheal (i.t.) Administration of 1 V270 and Collection of Bronchial Alveolar Lavage (BAL)

Mice were anesthetized and intratracheally (i.t.) or intranasally (i.n.) administered with the indicated doses of 1V270 dispersed in 50 μL PBS which forms small (100-150 nm particles). The same solution without the drug was used as a vehicle control. Preliminary experiment with vital dye showed that both i.n. and i.t. delivery methods led to pulmonary dispersal of the drug solution. At 6, 24, 48, and 72 h after administration, mice were sacrificed and the sera, BAL fluid and lungs were collected as described previously [17]. The levels of cytokines in BAL fluids and sera were determined by Luminex bead assay (Life Technologies). Total BAL cell numbers were determined using a Guava Personal Cytometer (EMD Millipore, Billerica, Mass.). Differential cell counts were morphologically determined after Wright-Giemsa staining. Lungs were fixed and stained with hematoxylin-eosin (H&E) by the UCSD Histology Shared Resource.

In Vivo Labeling of Dendritic Cells with Carboxyfluorescein Succinimidyl Ester (CFSE) and Flow Cytometric Analysis

CFSE was dissolved at 25 mM in DMSO and subsequently diluted to 8 mM in PBS. CFSE (50 μL) was i.n. administered to anesthetized mice as previously described [20]. Four to 5 h after the CFSE treatment, mice were i.t. administered with 1V270. 18 h after drug administration, mice were sacrificed and the cervical, mediastinal, mesenteric, and inguinal lymph nodes were collected. Lymph node cells were stained for CD11c or B220 to identify dendritic cells (DC), and B lymphcytes, respectively. The CFSE positive cells in the gated CD11c+ population and the B220+ population were enumerated using a FACSCanto flow cytometer (BD Bioscience, San Jose, Calif.) and analyzed using FlowJo software (Tree Star, Ashland, Oreg.).

### Efficacy Evaluation of 1V270 in Infectious Challenge Models

Three infection models were used to evaluate the immune protective efficacy of pulmonary 1V270 treatment. The viral infection models, utilizing H1N1 influenza and Venezuelan equine encephalitis virus (VEE), were performed at the Institute for Antiviral Research (Utah State University). The studies using Bacillus anthracis were performed at UCSD.

1) Influenza and VEE models: Female BALB/c mice were i.n. treated with 1 nmol 1V270 in 20 μL saline on days −3 and −1 before exposure to virus under anesthesia (ketamine/xylazine, 50/5 mg/kg by intraperitoneal [i.p.] administration). Mouse adapted influenza A/California/04/2009 (H1 N1) was kindly provided by Dr. Elena Govorkova (St. Jude Children's Research Hospital, Memphis, Tenn.) [21]. The Trinidad Donkey strain of VEE virus (strain NR-332) was obtained from BEI Resources (Manassas, Va.) and prepared in Vero cells as previously described [22]. On day 0, influenza H1N1 strain (90 μL of 104 50% cell culture infectious doses (CCID50)/mL per mouse) was administered i.n. to anesthetized mice [24]. VEE (0.1 mL of 5 CCID50/mL per mouse) was injectected subcutaneously [21, 23].

2) Anthrax model: Live spores from the Sterne strain of B. anthracis (pXO1+pXO2−) were prepared as previously described [18,24]. A/J mice were i.n. given 1V270 (1 nmol) or vehicle at 2-week intervals by the intranasal route for three times and challenged with heat-activated live spores 4 weeks after the last administration of 1V270. Mice were infected with 4×10⁶ CFU of live, heat-activated spores of the Sterne strain by i.n. administration, and survival was monitored daily for 30 days.

### Statistics

Data are presented as means±SEM or SD, as indicated. Student's t test was used to compare two groups. One-way ANOVA or the Mantel-Cox log-rank test was used for multiple group comparison. Kaplan-Meier survival curves and log-rank (Mantel-Cox) tests were performed for survival studies. Graph Pad Prism software version 5.0b (San Diego, Calif.) was used for analysis. A p value of <0.05 was considered to be statistically significant.

### Results

### Pulmonary Administration of 1 V270 Activates Local Innate Immune Response Without Induction of Systemic Immune Responses.

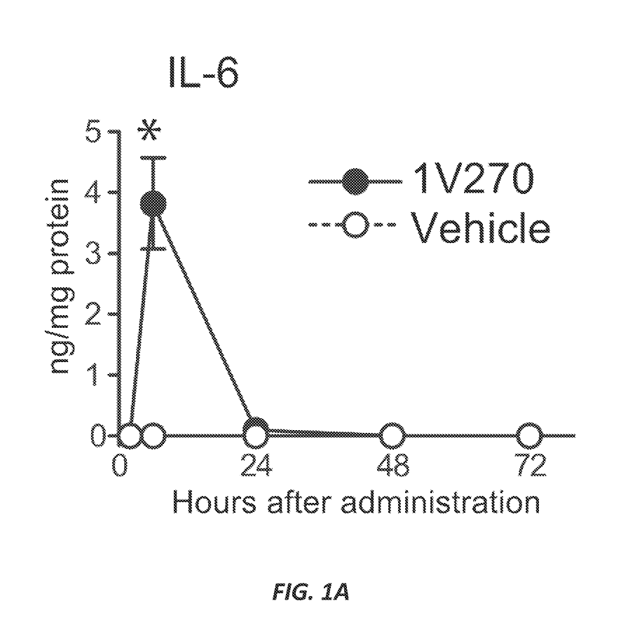

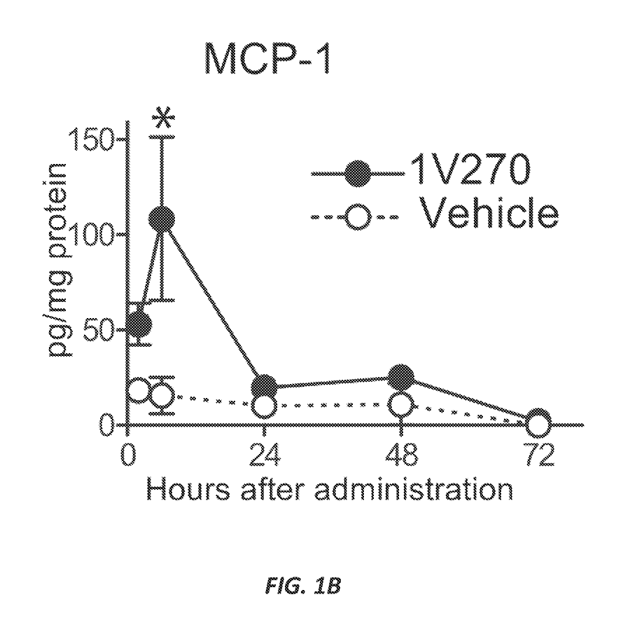

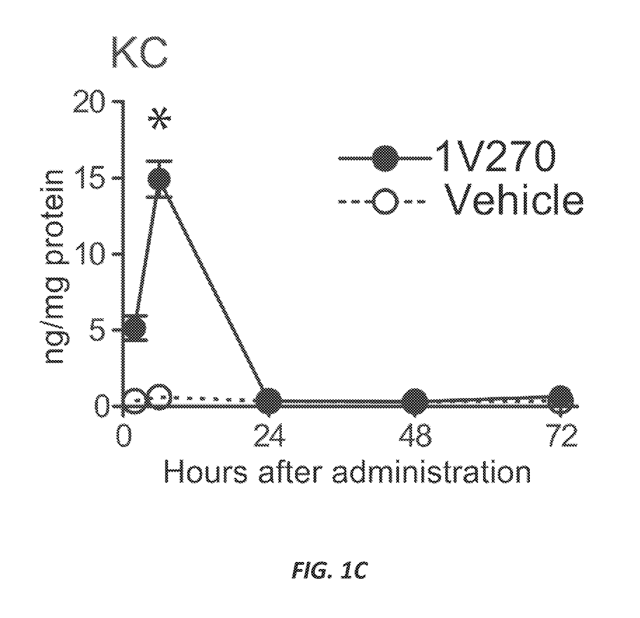

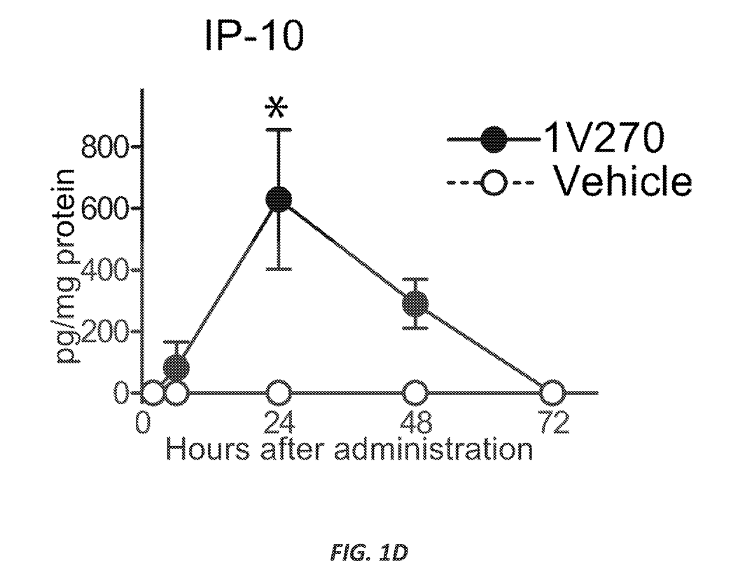

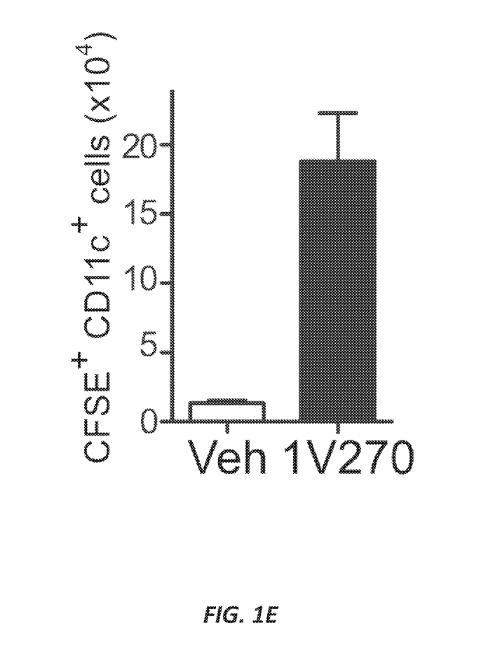

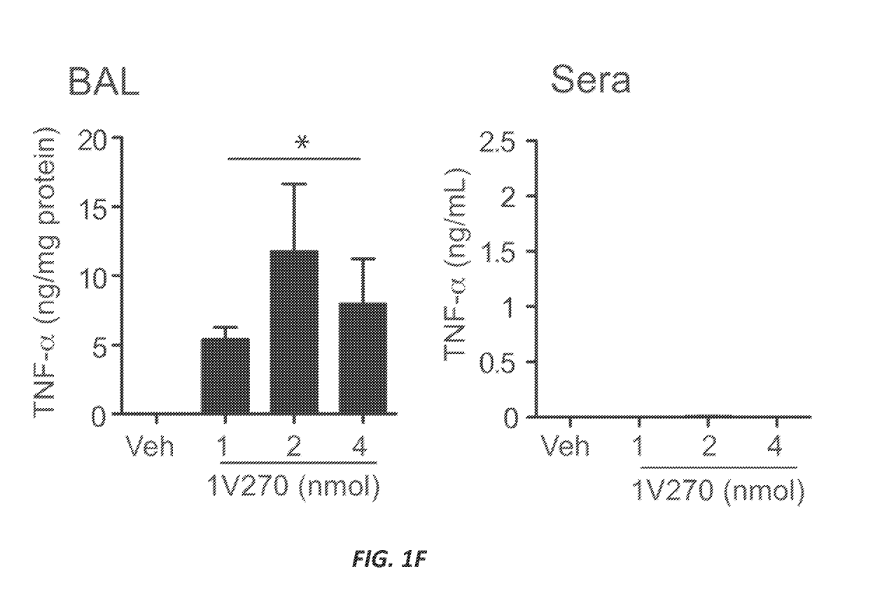

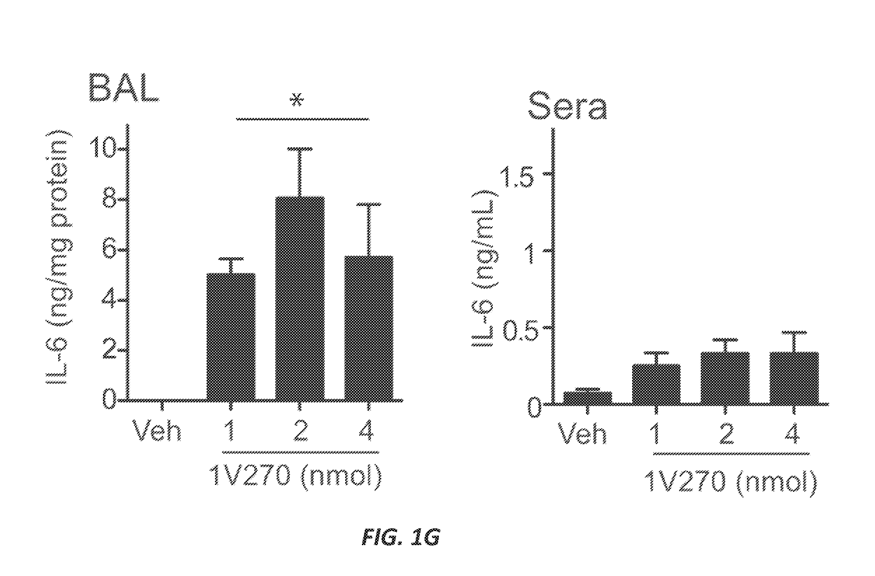

To characterize innate immune responses induced by pulmonary administration of 1V270, cytokines and chemokines in BAL fluids were monitored for up to 72 h after drug delivery. IL-6, monocyte chemoattractant protein-1 (MCP-1), keratinocyte chemoattractants (KC), and interferon gamma-induced protein 10 (IP-10) in BAL fluids were measured (FIG. 1). IP-10 was used as a surrogate marker of type 1 IFN induction [25].

IL-6, MCP-1 and KC significantly increased in 1V270-administered mice 6 h post-treatment (FIGS. 1A, 1B and 1C). IP-10 induction was peaked at 24 h and declined to baseline levels 72 h post treatment (FIG. 1D).

To evaluate the effect of 1V270 on DC activation and migration, the accumulation of CFSE labeled CD11c+ cells in the draining mediastinal lymph nodes 24 h after drug administration (FIG. 1E). The pulmonary 1V270 treatment enhanced CD11c+ DC migration to the mediastinal lymph nodes (FIG. 1E).

The principal adverse effect of systemic TLR7 ligand administration is a “cytokine syndrome” attributable by TNFα and related inflammatory mediators [26]. Hence, we compared the levels of TNFα and other pro-inflammatory cytokines in BAL fluid and in sera 24 h after pulmonary administration of 1V270. Notably, pulmonary 1V270 treatment elicited only a minimal insignificant increase in TNFα and IL-6 in sera up to a 4 nmol dose, which is 4 times the effective drug concentration for pulmonary protection (vide infra) (FIGS. 1F and 1G).

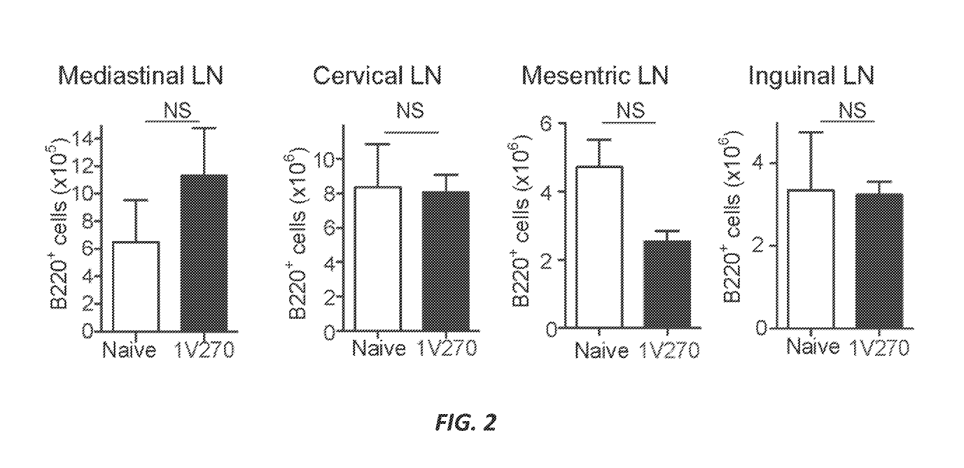

One of the significant adverse effects associated with the FDA-approved small molecule TLR7 ligand, imiquimod (IMQ), is lymphocytosis/plasmacytosis due to TLR7 activation of B cells [27]. To study the potential influence of pulmonary 1V270 on lymphoid organs, the B cell numbers in the cervical, mediastinal, mesenteric, and inguinal lymph nodes were compared (FIG. 2). 1V270 treatment did not significantly increase of B cell number in the draining medistinal lymph nodes (FIG. 2), nor in the cervical, mesenteric and inguinal nodes (FIG. 2).

### Pulmonary 1 V270 Treatment Causes Minimal Inflammation of the Lung Parenchyma and No Other Discernible Adverse Effects

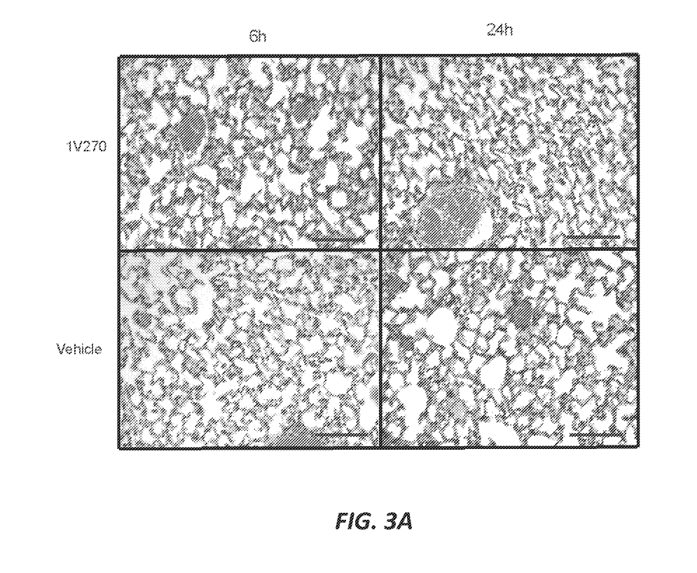

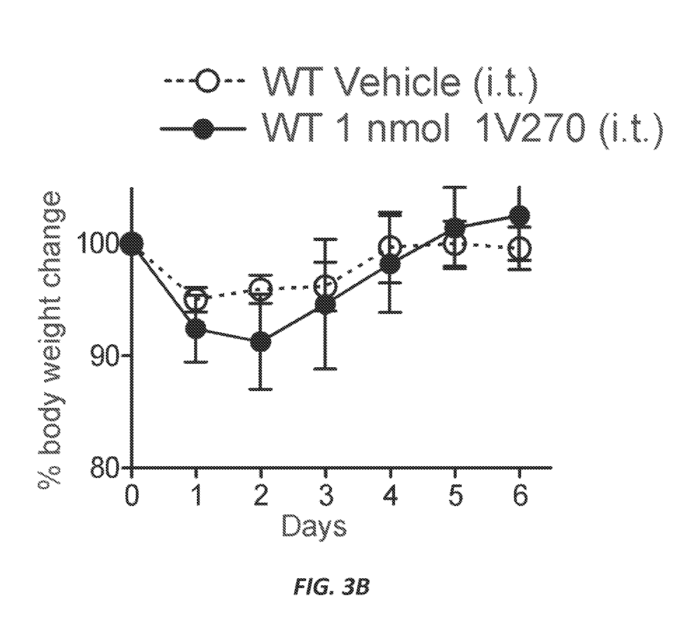

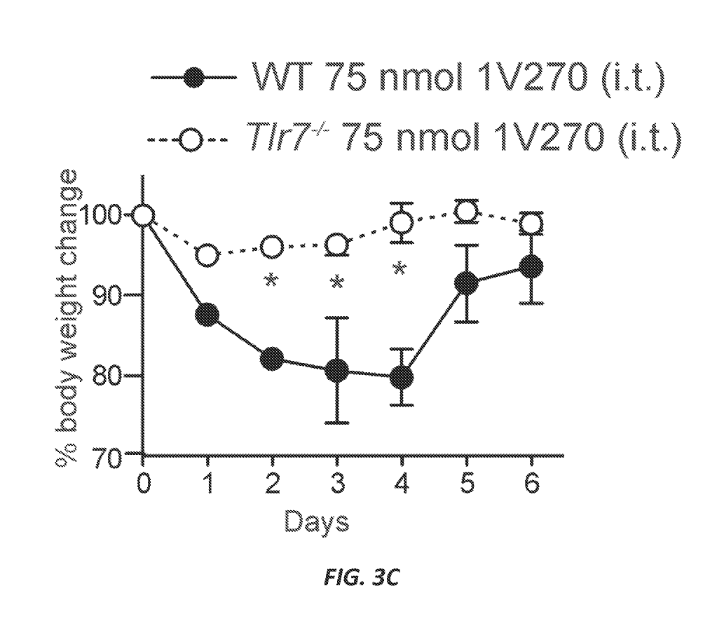

To evaluate whether local induction of proinflammatory cytokines and chemokines by 1V270 could cause pulmonary edema or interstitial inflammation, histological changes in the lung were evaluated 6 and 24 h post treatment. At both time points, IV270 treated lung sections did not show inflammatory cell infiltration into the lung parenchyma (FIG. 3A).

It was previously reported that high doses of an unconjugated TLR7 agonists caused anorexic behavior after both intraperitoneal and pulmonary administration [28]. In contrast, the effective dose (1 nmoles) of the phospholipid conjugated 1V270 did not cause significant weight loss compared to vehicle treated mice (FIG. 3B). Indeed, significant anorexia was only seen at the maximum tolerated dose (MTD) of 75 nmol/animal. This effect was entirely TLR7 dependent since TLR7 null mice did not lose weight after 1V270 treatment. Thus 1V270 does not apparently have off target toxic effects at dosages that induce immune responses in the lung.

### Pulmonary Administration of 1 V270 Promotes Neutrophil Infiltration in BAL Fluids in a TLR7/MyD88 Dependent Manner

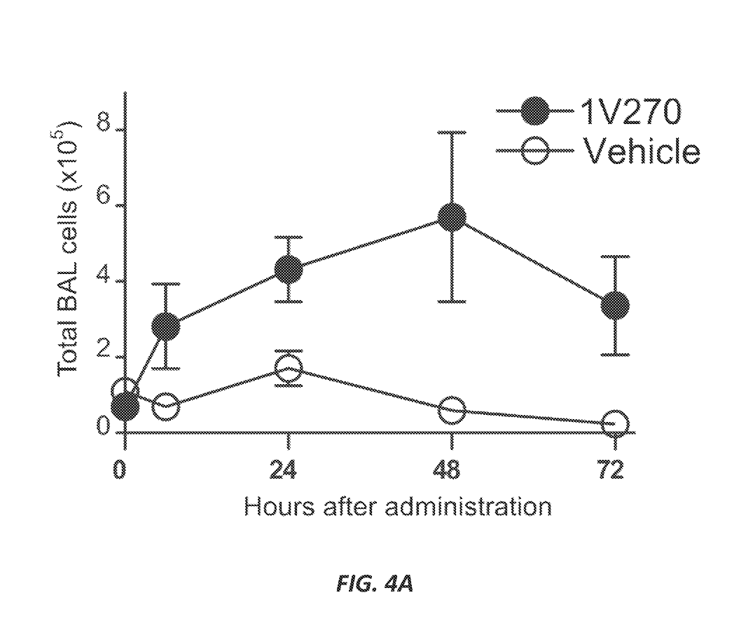

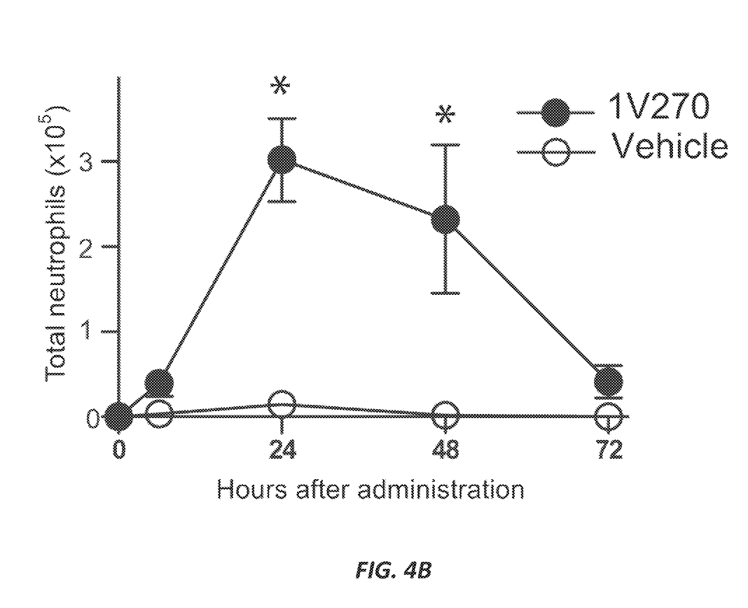

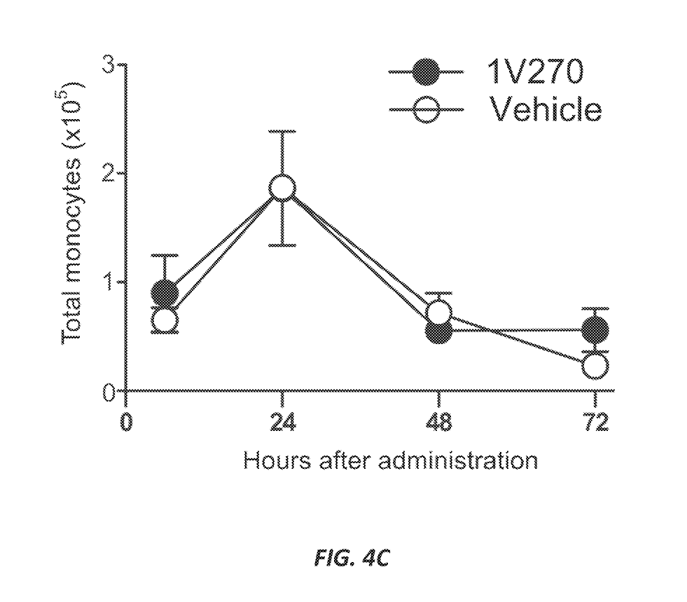

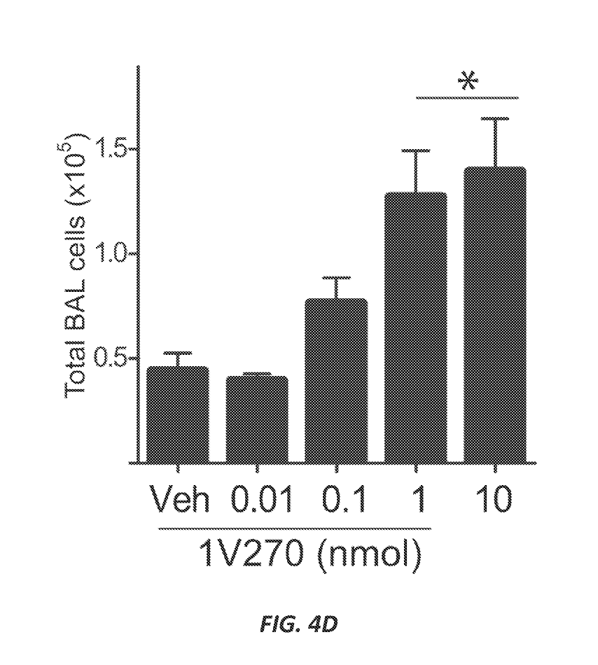

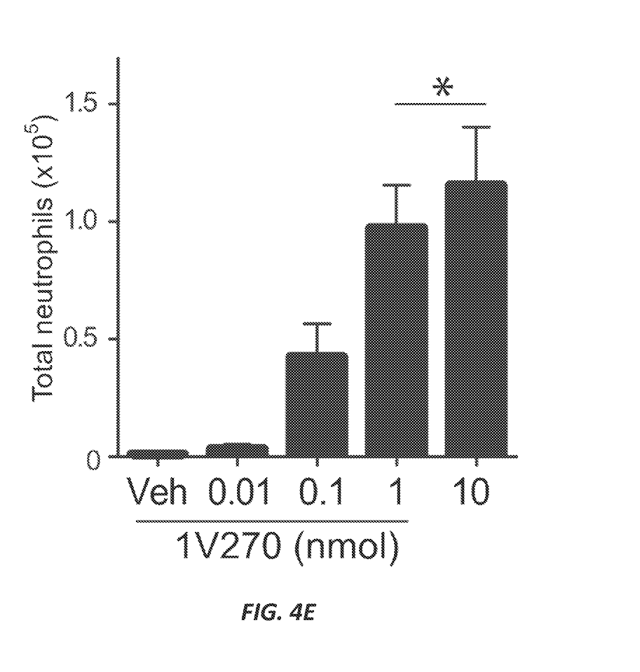

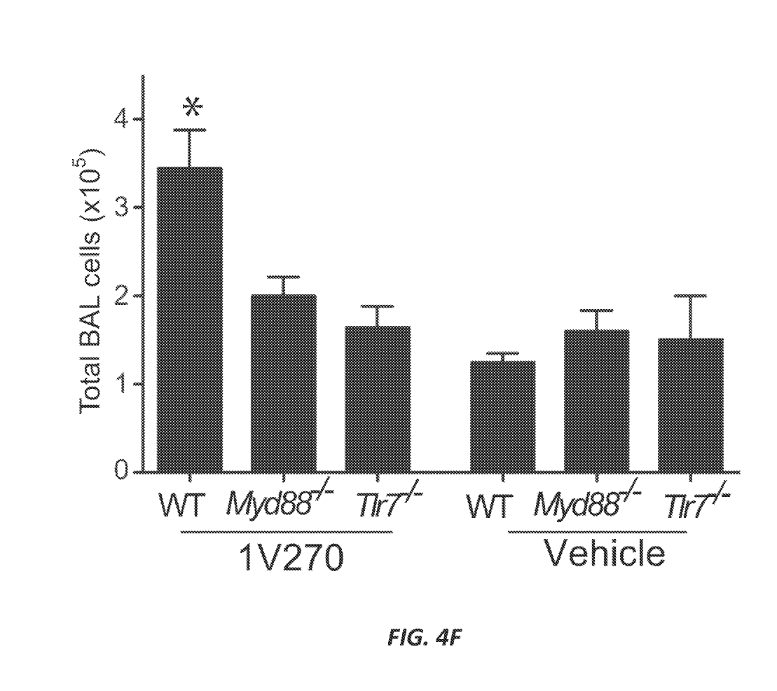

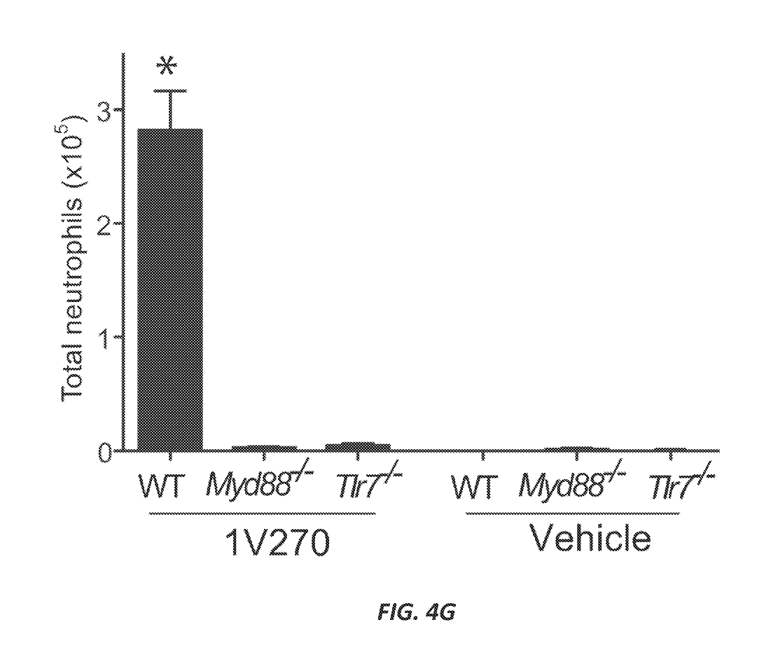

To further characterize the local innate immune response induced by pulmonary administration of 1V270, the cellular composition of the BAL fluids was monitored for 72 h after 1 nmol drug administration (FIG. 4).

Total cell numbers in the BAL fluids increased over 48 h and declined to near baseline levels 72 h post pulmonary administration (FIG. 4A). A significant increase was observed in the neutrophil population 24 h and 48 h after treatment (FIG. 4B), whereas mononuclear cells were slightly elevated in both 1V270- and vehicle-treated mice at 24 h after treatment (FIG. 4C). The neutrophil influx at 24 h was dose responsive between 0.1 to 10 nmol 1V270 (FIGS. 4c and 4d).

To confirm that the transient neutrophil accumulation induced by pulmonary administration of 1V270 is TLR7-MyD88 signaling pathway dependent, MyD88 or TLR7 null mice were treated (FIGS. 4E and 4F). 1V270 induced cell infiltration and neutrophil recruitment to BAL fluids was diminished in the two knockout strains (FIGS. 4E and 4F).

Pulmonary Treatment with 1 V270 Protects Mice from Infection

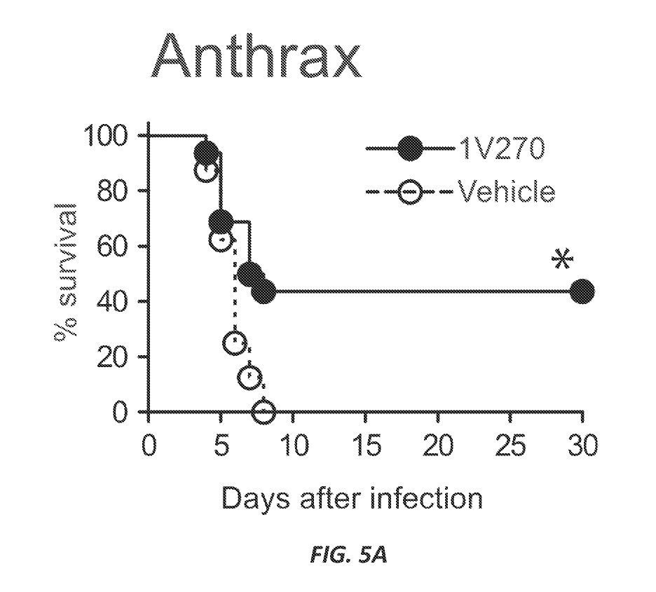

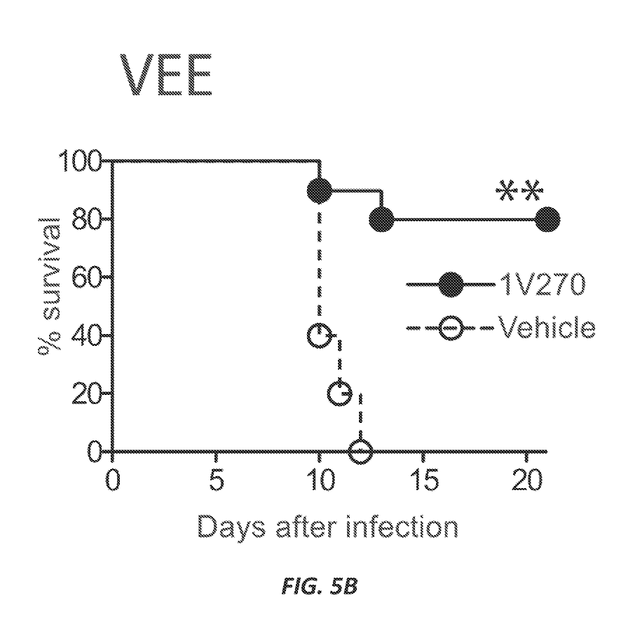

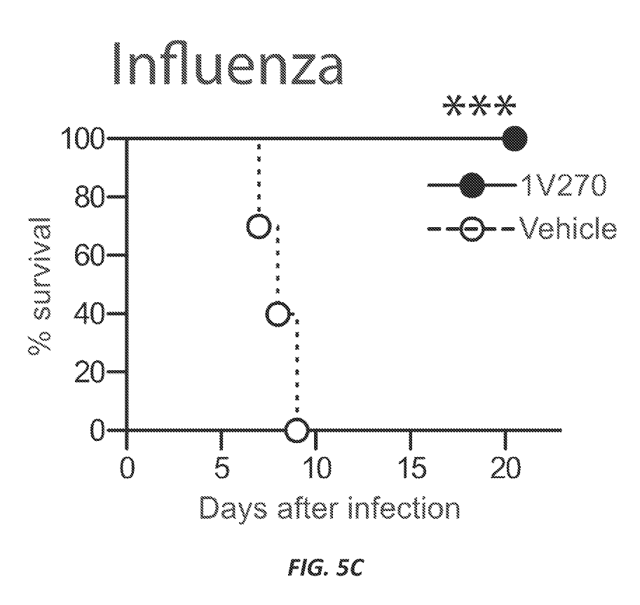

To evaluate the ability of pulmonary 1V270 to protect mice from pathogens selected NIAID Biodefense Category A, B, and C Priority Pathogens: inhalation anthrax, Venezuelan equine encephalitis (VEE) and inhalation H1N1 influenza were evaluated. To test the efficacy of pulmonary 1V270 treatment in a model of inhalation anthrax, NJ mice were given 1V270 three times at 2 week intervals. Four weeks after the last dose, the 1V270- or vehicle-treated mice were challenged i.n. with live Bacillus anthracis spores. Survival was monitored for 30 days. Forty % of the mice treated with 1V270 survived at least 30 days, while control mice were all dead by day 7 (p<0.05, FIG. 5A).

In the VEE infection model, the virus infects through the subcutaneous route and disperses in the lungs, blood and spleen prior to entering the central nervous system through the nasal olfactory nerves [29,30]. In this situation, innate immune stimulation in the nasal and respiratory tissues might prevent lethal encephalitis. To test this hypothesis, BALB/c mice received i.n treatments with 1V270 on days −3 and −1 before challenge with VEE virus subcutaneously. Eighty % of 1V270 treated mice were protected from encephalitis while all control mice died by 12 days after infection (FIG. 5B). Using the same prophylactic protocol, 1V270 protected 100% of mice from lethal H1N1 pulmonary influenza infection (FIG. 5).

### Discussion

The pulmonary route of infection is of particular relevance in terms of bioterrorism since it is a quick way to disperse an infectious agent to a susceptible population. The data presented here demonstrates that pulmonary delivery of a phospholipid conjugated TLR7 ligand, 1V270, activated local innate immune responses, without causing a systemic cytokine syndrome and without damaging the pulmonary parenchymal tissues. This treatment completely prevented lethal pulmonary infection by influenza virus, and protected 40% of exposed animals from inhaled anthrax. This treatment was also effective in prevention of death from VEE virus after s.c challenge.

The safety profile of pulmonary 1V270 treatment was determined. Prophylactic administration of ligands for TLR2, TLR3, TLR4, and TLR9 has been reported to reduce the severity of pulmonary influenza infection [4,6,7,9,10,13,14]. In spite of the efficacy of the TLR activators, these drugs have raised safety concerns that have impeded their clinical development [16]. Among TLR ligands, imiquimod is an FDA-approved TLR7 agonist for the topical treatment of papilloma virus infections [31]. However, imiquimod can cause systemic cytokine release, and also exhibits significant off target effects that are independent of TLR7 and TLR8 [32]. The data presented here shows that 1V270, a phospholipid conjugated TLR7 ligand, provides several safety advantages compared to most other TLR agonists. First, pharmacodynamic data indicate that immune activation by pulmonarylV270 is confined to the respiratory tract. Effective doses of the drug did not significantly increase cytokine levels in the blood and did not cause anorexia. Second, pulmonary 1V270 did not induce B cell proliferation in secondary lymphoid organs. Third, the cytokine induction and cell infiltration in BAL induced by 1V270 was transient and was not associated with lung interstitial inflammation. The maximum tolerated dose (MTD) of 1V270 administered by the pulmonary route was 75 nmol/animal, which is 75 times higher than the effective dose of 1 nmol. Moreover, the body weight loss induced by the maximum tolerated dose (MTD) was entirely TLR7 dependent, and was not attributable to off-target toxicity [28]. In humans, TLR7 expression is primarily limited to plasmacytoid DCs (pDCs) and to activated B cells under normal conditions. TLR2, 3 and 4 are expressed in a broad range of cell types [33,34]. The limited expression pattern of TLR7 receptor may also prevent excessive immune reactions by pulmonary 1V270. The inhalation of TLR9 activators has been reported to be safe in humans at dose that stimulate innate immune responses [35]. Taken together the data indicate that 1V270, given by the pulmonary route, should also display an excellent safety profile in humans.

The efficacy of pulmonary 1V270 treatment as an immune protectant against a range of infectious pathogens was determined. Pulmonary 1V270 treatment broadly activated local innate immune responses, inducing both neutrophil recruitment and IP-10, a surrogate marker of type 1 IFN, in the BAL fluids. Local or systemic administration of type 1 IFN mediates immune defenses against RNA viruses including influenza [36,37]. A recent report indicated that neutrophils also play a protective role in severe influenza infection in mice [38]. Consistent with it effects on both interferon and neutrophils, pulmonary 1V270 treatment protected 100% of mice from lethal H1N1 influenza.

The 1V270 treatment also protected mice from pulmonary infection by inhaled Bacillus anthracis spores, confirming earlier results with an albumin conjugated TLR7 ligand [39,40]. In part, the induction of type 1 IFN may be involved in protection from anthrax because intranasal administration of the type 1 IFN inducer, Poly-ICLC was also reported to protect animals from inhaled anthrax [39]. However, the 1V270 treatment caused activation of pulmonary dendritic cells that migrated to the regional lymph nodes. It seems likely that the persistent immune protection induced by 1V270 may be due to effects on activated DCs and lymphocytes. To test the efficacy of pulmonary 1V270 treatment in a model utilizing a route other than airway challenge, the s.c. route of infection in a VEE virus model was used. Because natural infection by the VEE virus is mediated by mosquito bites, the subcutaneous challenge model is well characterized in mice [41,42]. In this model, virus replication in the draining lymph node is detectable within 3 h and the peak of viremia is observed at 12 h. Then, the virus replicates in the nasal epithelium and enters the central nervous system through sensory neurons in the nasal olfactory neuroepithelium. Death ensues 7 to 10 days after infection [42-47]. Although systemic type 1 IFN shows protective effects on s.c. infection by VEE virus [41,48], pulmonary 1V270 did not induce systemic cytokines. However it is very likely that intranasal treatment with1V270 induced potent innate immune responses in the nasal passage, that inhibited the entry of the virus into the olfactory neuroepithelium and the brain. It is also possible that activated DCs play a role in limiting viral spread.

The data presented here show that pulmonary administration of a phospholipid conjugated TLR7 agonist, 1V270, activated local innate immune responses, and provided protection from multiple infectious diseases. The immune activation by pulmonary 1V270 treatment did not induce systemic cytokine release, or B cell proliferation. Prophylactic administration of 1V270 could be a potential biodefense agent to increase host resistant to pulmonary pathogens with minimal side effects.

### REFERENCES

1 Kurt-Jones E A, Popova L, Kwinn L, Haynes L M, Jones L P, Tripp R A, Walsh E E, Freeman M W, Golenbock D T, Anderson L J, Finberg R W: Pattern recognition receptors tlr4 and cd14 mediate response to respiratory syncytial virus. Nat Immunol 2000; 1:398-401.
2 Kawai T, Akira S: Toll-like receptors and their crosstalk with other innate receptors in infection and immunity. Immunity 2011; 34:637-650.
3 Williams A E, Edwards L, Humphreys I R, Snelgrove R, Rae A, Rappuoli R, Hussell T: Innate imprinting by the modified heat-labile toxin of escherichia coli (Itk63) provides generic protection against lung infectious disease. J Immunol 2004; 173:7435-7443.
4 Hammerbeck D M, Burleson G R, Schuller C J, Vasilakos J P, Tomai M, Egging E, Cochran F R, Woulfe S, Miller R L: Administration of a dual toll-like receptor 7 and toll-like receptor 8 agonist protects against influenza in rats. Antiviral Res 2007; 73:1-11.
5 Hutchens M A, Luker K E, Sonstein J, Nunez G, Curtis J L, Luker G D: Protective effect of toll-like receptor 4 in pulmonary vaccinia infection. PLoS Pathog 2008; 4:e1000153.
6 Wong J P, Christopher M E, Viswanathan S, Karpoff N, Dai X, Das D, Sun L Q, Wang M, Salazar A M: Activation of toll-like receptor signaling pathway for protection against influenza virus infection. Vaccine 2009; 27:3481-3483.
7 Abdul-Careem M F, Firoz Mian M, Gillgrass A E, Chenoweth M J, Barra N G, Chan T, Al-Garawi A A, Chew M V, Yue G, van Roojen N, Xing Z, Ashkar A A: Fimh, a tlr4 ligand, induces innate antiviral responses in the lung leading to protection against lethal influenza infection in mice. Antiviral Res 2011; 92:346-355.
8 Nasirudeen A M, Wong H H, Thien P, Xu S, Lam K P, Liu D X: Rig-i, mda5 and tlr3 synergistically play an important role in restriction of dengue virus infection. PLoS Negl Trop Dis 2011; 5:e926.
9 Shinya K, Okamura T, Sueta S, Kasai N, Tanaka M, Ginting T E, Makino A, Eisfeld A J, Kawaoka Y: Toll-like receptor pre-stimulation protects mice against lethal infection with highly pathogenic influenza viruses. Virol J 2011; 8:97.
10 Shinya K, Ito M, Makino A, Tanaka M, Miyake K, Eisfeld A J, Kawaoka Y: The tlr4-trif pathway protects against h5n1 influenza virus infection. J Virol 2012; 86:19-24.
11 Beckett E L, Phipps S, Starkey M R, Horvat J C, Beagley K W, Foster P S, Hansbro P M: Tlr2, but not tlr4, is required for effective host defence against chlamydia respiratory tract infection in early life. PLoS One 2012; 7:e39460.
12 St Paul M, Mallick A I, Read L R, Villanueva A I, Parvizi P, Abdul-Careem M F, Nagy E, Sharif S: Prophylactic treatment with toll-like receptor ligands enhances host immunity to avian influenza virus in chickens. Vaccine 2012; 30:4524-4531.
13 Tuvim M J, Gilbert B E, Dickey B F, Evans S E: Synergistic tlr2/6 and tlr9 activation protects mice against lethal influenza pneumonia. PLoS One 2012; 7:e30596.
14 Tan A C, Mifsud E J, Zeng W, Edenborough K, McVernon J, Brown L E, Jackson D C: Intranasal administration of the tlr2 agonist pam2cys provides rapid protection against influenza in mice. Mol Pharm 2012; 9:2710-2718.
15 Roquilly A, Broquet A, Jacqueline C, Gautreau L, Segain J P, de Coppet P, Caillon J, Altare F, Josien R, Asehnoune K: Tlr-4 agonist in post-haemorrhage pneumonia: Role of dendritic and natural killer cells. Eur Respir J 2013; May 16. [Epub ahead of print]
16 Engel A L, Holt G E, Lu H: The pharmacokinetics of toll-like receptor agonists and the impact on the immune system. Expert Rev Clin Pharmacol 2011; 4:275-289.
17 Chan M, Hayashi T, Kuy C S, Gray C S, Wu C C, Corr M, Wrasidlo W, Cottam H B, Carson D A: Synthesis and immunological characterization of toll-like receptor 7 agonistic conjugates. Bioconjug Chem 2009; 20:1194-1200.
18 Wu C C, Hayashi T, Takabayashi K, Sabet M, Smee D F, Guiney D D, Cottam H B, Carson D A: Immunotherapeutic activity of a conjugate of a toll-like receptor 7 ligand. Proc Natl Acad Sci USA 2007; 104:3990-3995.
19 Agassandian M, Mallampalli R K: Surfactant phospholipid metabolism. Biochim Biophys Acta 2013; 1831:612-625.
20 Belz G T, Smith C M, Kleinert L, Reading P, Brooks A, Shortman K, Carbone F R, Heath W R: Distinct migrating and nonmigrating dendritic cell populations are involved in mhc class i-restricted antigen presentation after lung infection with virus. Proc Natl Acad Sci USA 2004; 101:8670-8675.
21 Smee D F, von Itzstein M, Bhatt B, Tarbet EB: Exacerbation of influenza virus infections in mice by intranasal treatments and implications for evaluation of antiviral drugs. Antimicrob Agents Chemother 2012; 56:6328-6333.
22 Julander J G, Bowen R A, Rao J R, Day C, Shafer K, Smee D F, Morrey J D, Chu C K: Treatment of venezuelan equine encephalitis virus infection with (−)-carbodine. Antiviral Res 2008; 80:309-315.
23 Bennett A M, Elvin S J, Wright A J, Jones S M, Phillpotts R J: An immunological profile of balb/c mice protected from airborne challenge following vaccination with a live attenuated venezuelan equine encephalitis virus vaccine. Vaccine 2000; 19:337-347.
24 Sabet M, Cottam H B, Guiney D G: Modulation of cytokine production and enhancement of cell viability by tlr7 and tlr9 ligands during anthrax infection of macrophages. FEMS Immunol Med Microbiol 2006; 47:369-379.
25 Holzinger D, Jorns C, Stertz S, Boisson-Dupuis S, Thimme R, Weidmann M, Casanova J L, Haller O, Kochs G: Induction of mxa gene expression by influenza a virus requires type i or type iii interferon signaling. J Virol 2007; 81:7776-7785.
26 Burkhart C G: Reports of immunologic reactions to imiquimod: An assessment of actinic keratoses and treatment concerns in this era of litigation lotto. Dermatol Online J 2005; 11:40.
27 Richwald G A: Imiquimod. Drugs Today (Barc) 1999; 35:497-511.
28 Hayashi T, Cottam H B, Chan M, Jin G, Tawatao R I, Crain B, Ronacher L, Messer K, Carson D A, Corr M: Mast cell-dependent anorexia and hypothermia induced by mucosal activation of toll-like receptor 7. American journal of physiology 2008; 295:R123-132.
29 Ryzhikov A B, Tkacheva N V, Sergeev A N, Ryabchikova E I: Venezuelan equine encephalitis virus propagation in the olfactory tract of normal and immunized mice. Biomed Sci 1991; 2:607-614.
30 Schafer A, Brooke C B, Whitmore A C, Johnston R E: The role of the blood-brain barrier during venezuelan equine encephalitis virus infection. J Virol 2011; 85:10682-10690.
31 Stern P L, van der Burg S H, Hampson I N, Broker T R, Fiander A, Lacey C J, Kitchener H C, Einstein M H: Therapy of human papillomavirus-related disease. Vaccine 2012; 30 Suppl 5:F71-82.
32 Schon M P, Schon M: Imiquimod: Mode of action. Br J Dermatol 2007; 157 Suppl 2:8-13.
33 Kovach M A, Standiford T J: Toll like receptors in diseases of the lung. Int Immunopharmacol 2011; 11:1399-1406.
34 Schill T, Schon M P, Pletz N, Emmert S, Schon M: Stimulation of pulmonary immune responses by the tlr2/6 agonist malp-2 and effect on melanoma metastasis to the lung. Exp Dermatol 2012; 21:91-98.
35 Campbell J D, Cho Y, Foster M L, Kanzler H, Kachura M A, Lum J A, Ratcliffe M J, Sathe A, Leishman A J, Bahl A, McHale M, Coffman R L, Hessel E M: Cpg-containing immunostimulatory DNA sequences elicit tnf-alpha-dependent toxicity in rodents but not in humans. J Clin Invest 2009; 119:2564-2576.
36 Beilharz M W, Cummins J M, Bennett A L: Protection from lethal influenza virus challenge by oral type 1 interferon. Biochem Biophys Res Commun 2007; 355:740-744.
37 Garcia-Sastre A, Biron C A: Type 1 interferons and the virus-host relationship: A lesson in detente. Science 2006; 312:879-882.
38 Tate M D, loannidis L J, Croker B, Brown L E, Brooks A G, Reading P C: The role of neutrophils during mild and severe influenza virus infections of mice. PLoS One 2011; 6:e17618.
39 Walberg K, Baron S, Poast J, Schwartz B, Izotova L, Pestka S, Peterson J W: Interferon protects mice against inhalation anthrax. J Interferon Cytokine Res 2008; 28:597-601.
40 Cote C K, Van Rooijen N, Welkos S L: Roles of macrophages and neutrophils in the early host response to bacillus anthracis spores in a mouse model of infection. Infect Immun 2006; 74:469-480.
41 Lukaszewski R A, Brooks TJ: Pegylated alpha interferon is an effective treatment for virulent venezuelan equine encephalitis virus and has profound effects on the host immune response to infection. J Virol 2000; 74:5006-5015.
42 Ryzhikov A B, Ryabchikova E I, Sergeev A N, Tkacheva N V: Spread of venezuelan equine encephalitis virus in mice olfactory tract. Arch Virol 1995; 140:2243-2254.
43 Aronson J F, Grieder F B, Davis N L, Charles P C, Knott T, Brown K, Johnston R E: A single-site mutant and revertants arising in vivo define early steps in the pathogenesis of venezuelan equine encephalitis virus. Virology 2000; 270:111-123.
44 Vogel P, Abplanalp D, Kell W, Ibrahim M S, Downs M B, Pratt W D, Davis K J: Venezuelan equine encephalitis in balb/c mice: Kinetic analysis of central nervous system infection following aerosol or subcutaneous inoculation. Arch Pathol Lab Med 1996; 120:164-172.
45 Charles P C, Walters E, Margolis F, Johnston R E: Mechanism of neuroinvasion of venezuelan equine encephalitis virus in the mouse. Virology 1995; 208:662-671.
46 Grieder F B, Davis N L, Aronson J F, Charles P C, Sellon D C, Suzuki K, Johnston R E: Specific restrictions in the progression of venezuelan equine encephalitis virus-induced disease resulting from single amino acid changes in the glycoproteins. Virology 1995; 206:994-1006.
47 Gleiser C A, Gochenour W S, Jr., Berge T O, Tigertt W D: The comparative pathology of experimental venezuelan equine encephalomyelitis infection in different animal hosts. J Infect Dis 1962; 110:80-97.
48 O'Brien L, Perkins S, Williams A, Eastaugh L, Phelps A, Wu J, Phillpotts R: Alpha interferon as an adenovirus-vectored vaccine adjuvant and antiviral in venezuelan equine encephalitis virus infection. J Gen Virol 2009; 90:874-882.

### Example 2

The effectiveness of intranasal administration compound 1V270 against West Nile virus infection in mice was evaluated to ascertain if this compound, even when administered by intranasal route, can provide protection against a virus infection that is devoid of any significant respiratory involvement.

### Materials

### Animals

C57BL/6 mice, greater than 7 weeks of age, were used (17.4 g±0.9) (Charles River Laboratories). Animals were randomized to treatment groups. This study was conducted in accordance with and with the approval of the Institutional Animal Care and Use Committee of Utah State University dated 24 Sep. 2012. The work was done in the AAALAC-accredited Laboratory Animal Research Center of Utah State University. The U. S. Government (National Institutes of Health) approval was renewed 28 Feb. 2014 (Assurance no. A3801-01) in accordance with the National Institutes of Health Guide for the Care and Use of Laboratory Animals (Revision; 2010).

### Viruses

WN02 isolate (KERN) dated April 2010 was used. The Kern isolate was shipped to USU by Battelle Laboratories (KERN 515: Mosquito, 10/05/07, Kern County, Calif., TVP 10799, BBRC lot # WNVKERN515-01). Viral cultures were first confirmed to be mycoplasma-free using the PlasmoTest kit (cat #rep-pt2, InvivoGen). MA-104 cells at 95% confluence were inoculated with 0.01 mL of stock viruses. Four days after inoculation, cell culture supernatants for the WN02 KERN strain were collected and centrifuged at 15,000 rpm at room temp. The supernatants were then concentrated using Amicon stirred cells filter per the manufacturer's instructions (Cat#5124). The stock was assayed to be 2.5×10⁶ pfu/mL.

### Compound

Endotoxin-free saline (for hospital-use) and water was used to prepare a solution of 1V-270. A volume of 0.100 mL of 40× stock 1V-270 was diluted into 2.0 mL of water by adding to water drop-wise. It was sonicated 10 min at room temperature. On -3day, half the volume (1.0 mL) was diluted to 2.0 mL in saline. On −1 day, the same was done with the remaining half of the 2× solution (1.0 mL).

### Methods

### Experiment Design

Adult female C57BL/6 mice were be randomized to the treatment groups (Table 1) and inoculated intranasally (i.n.) with 1V270 (TMX-201) or placebo at days −3 or −1 before viral challenge. At day 0, mice were inoculated subcutaneously (s.c.) with West Nile Virus (WNV). Mortality and weight changes were monitored.

TABLE 1

| Animals | | | | | | |
|---|---|---|---|---|---|---|
| per | | Infect | | | | |
| cage | Group # | y or n | Compound | Dosage | | Treatment Schedule |
| 5 | 1A | y | TMX-201 | 1 | nmol | one i.n. instillation at −3 d and −1 d |
| 5 | 1B | y | TMX-201 | 1 | nmol | one i.n. instillation at −3 d and −1 d |
| 5 | 1C | y | TMX-201 | 1 | nmol | one i.n. instillation at −3 d and −1 d |
| 5 | 3A | y | TMX-201 | 1 | nmol | one i.n. instillation at −1 d |
| 5 | 3B | y | TMX-201 | 1 | nmol | one i.n. instillation at −1 d |
| 5 | 3C | y | TMX-201 | 1 | nmol | one i.n. instillation at −1 d |
| 5 | 5A | y | Placebo | — | | one i.n. instillation at −3 d and −1 d |
| 5 | 5B | y | Placebo | — | | one i.n. instillation at −3 d and −1 d |
| 5 | 5C | y | Placebo | — | | one i.n. instillation at −3 d and −1 d |
| 5 | 7A | y | Pfizer WNV Innovator | 10⁻² | dilution | one i.m. injection at −3 d |
| 5 | 7B | y | Pfizer WNV Innovator | 10⁻² | dilution | one i.m. injection at −3 d |
| 5 | 7C | y | Pfizer WNV Innovator | 10⁻² | dilution | one i.m. injection at −3 d |
| 2 | 2A | n | TMX-201 | 1 | nmol | one i.n. instillation at −3 d and −1 d |
| 2 | 4A | n | Pfizer WNV Innovator | 10⁻² | dilution | one i.m. injection at −3 d |

| | | | | | |
|---|---|---|---|---|---|
| 2 | 6A | n | Placebo | — | one i.n. instillation at −3 d and −1 d |

### Results

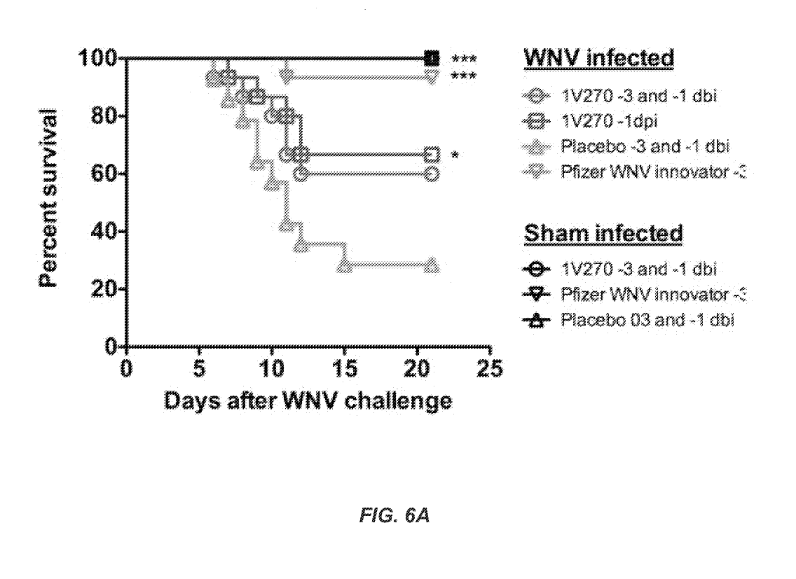

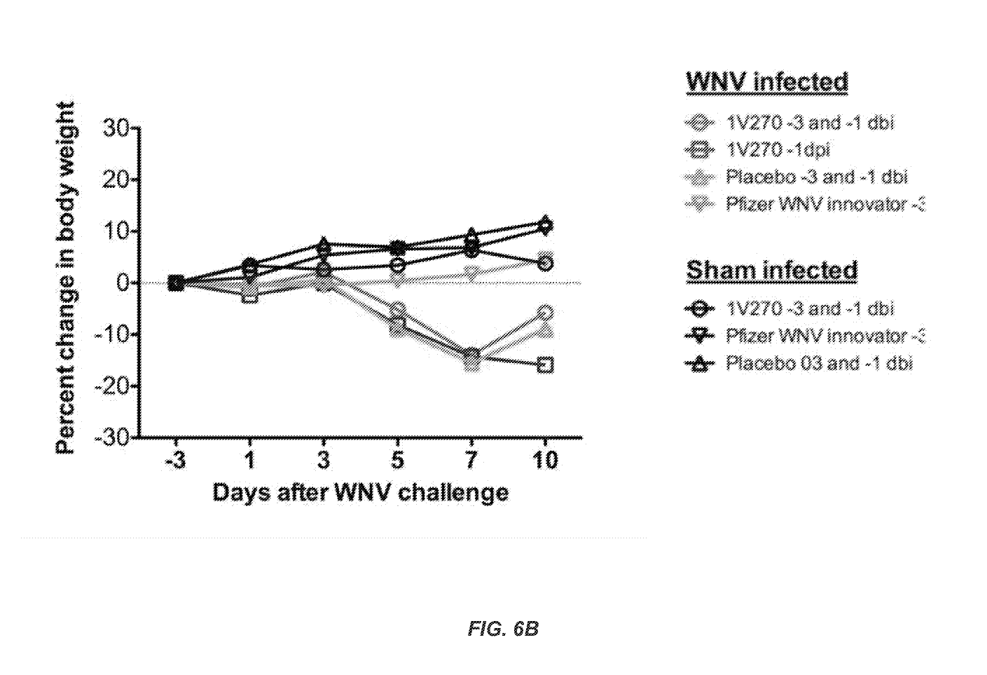

The immunomodulator 1V270(TMX-201) administered by intranasal instillation at a dosage of 1 nmol/mouse on day -1 was statistically improved compared to the placebo-treated mice (FIG. 6A) (P≦0.05). The survival curve for the other 1V270-group of mice treated at days −3 and −1 very closely resembled the mice treated only at day −1, except that the statistical analysis was not significant from the placebo-treated mice. Overall, survival of both groups of 1V270-treated mice appeared to be different from the placebo-treated mice. The Pfizer WNV innovator-vaccine positive control survival was clearly improved over the placebo-treated survival (P<0.001). There was no mortality in the sham-infected control groups.

The weight changes of the WNV-infected mice treated with the two 1V270-treated groups were similar to the placebo, infected group (FIG. 6B). The weight changes of the Pfizer WNV innovator, infected group were similar to the sham-infected control groups. Typically, weight change of infected mice is associated with efficacy of the treatment, however, that does not appear to be the case with this study.

### CONCLUSIONS

1. The immunomodulator 1V270 administered by intranasal instillation at a dosage of 1 nmol/mouse on day -1 was statistically improved compared to the placebo-treated mice.
2. Overall, survival of both groups of 1V270-treated mice appeared to be different from the placebo-treated mice.
3. The weight changes of the WNV-infected mice treated with the two 1V270-treated groups were similar to the placebo, infected group.

### Example 3

Vaccinia virus infects via the respiratory system. The effectiveness of intranasal administration compound 1V270 against vaccinia virus infection in mice was examined.

### Materials

### Animals

Female 14-16 g BALB/c mice were obtained from Charles River Laboratories (Wilmington, MA) for this investigation. The animals were maintained on standard rodent chow and tap water ad libitum. The animals were quarantined for at least 48 hours prior to use.

### Virus

Vaccinia virus (IHD strain) was purchased from the American Type Culture Collection (ATCC, Manassas, Va.). The virus was propagated in African green monkey kidney (MA-104) cells (MA Bioproducts, Walkersville, Md.) and titrated for lethality in mice. The infection model that used this particular virus strain has been published (1).

### Compounds

Preparation of 1V270 for intranasal instillation was as follows: The compound was initially diluted 1:20 in water and sonicated in a sonicating water bath for 10 minutes under low power (cool water circulated through the sonicating vessel to prevent the buildup of heat). Sonication was done within 60 minutes of using it for treatment. Then the material was diluted 1:2 in physiological saline before treatment of the mice, resulting in a 1:40 final dilution. When administered intranasally in a 50-pl volume, this equated to a dose of 0.3 or 1 nmol/mouse. The compound was made up fresh for each treatment. The antibodies were diluted so that a 0.1 ml intraperitoneal (i.p.) injection volume equated to 200 pg/mouse. Cidofovir was purchased from Bosche Scientific (New Brunswick, NJ). It was prepared in sterile saline.

### Methods

### Experiment Design

Mice were anesthetized by intraperitoneal (i.p.) injection of ketamine/xylazine (50/5 mg/kg) and then were exposed intranasally to a 50-μl suspension of vaccinia virus. The infectious inoculum of virus (approximately 10⁵ CCID₅₀/mouse) equated to approximately 3-4 50% mouse lethal challenge doses (MLD₅₀). There were 10 mice/group treated with test compounds and 20 placebos (treated with saline).

Intranasal treatments in a 50-μl volume with 1V270 and placebo saline were performed once a day at the indicated times in the table and figures. Mice were weighed individually every other day through day 21 of the infection. Animals whose body weight fell below 70% of starting weight were considered to have reached the endpoint of mortality, were humanely euthanized and eliminated from the study.

### Statistical Analysis

Survival curves were initially compared by the Mantel-Cox log-rank test, and statistical significance was found. Subsequently, pairwise comparisons of survival curves were made using the Gehan-Breslow-Wilcoxon test with Bonferroni corrected threshold of significance for the number of treatment groups evaluated. Survivors/total data in the table were evaluated by two-tailed Fisher's exact test. Mean day of death data in the table were evaluated by the Tukey-Kramer multiple comparisons test. Calculations were made using Instat 3.10 and Prism 6.0 software programs (GraphPad Software, San Diego, Calif.). Statistical comparisons were made between treated and placebo groups.

### Ethics Regulation of Laboratory Animals:

The study was conducted in accordance with the approval of the Institutional Animal Care and Use Committee of Utah State University dated 20 Sep. 2010 (expiration date 19 Sep. 2013). The work was done in the AAALAC-accredited Laboratory Animal Research Center of Utah State University.

### Results

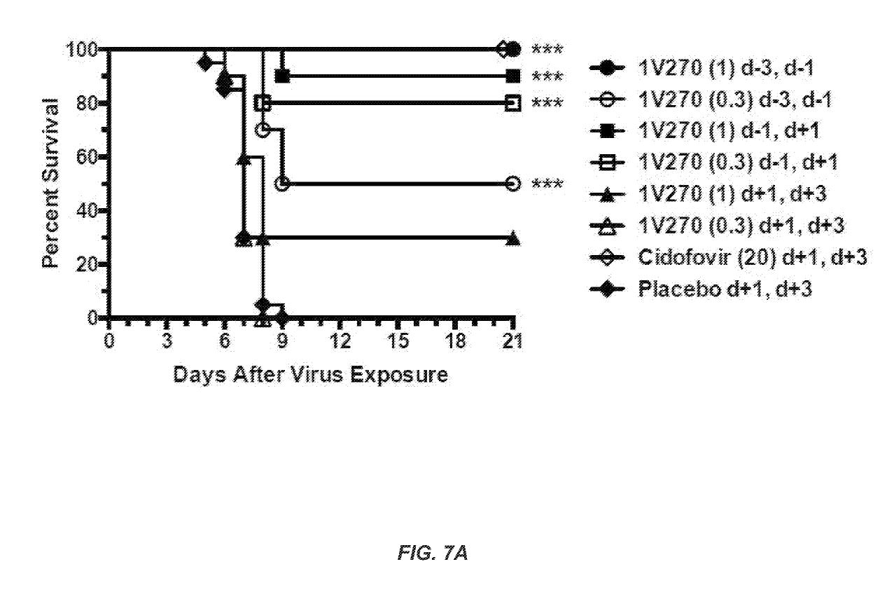

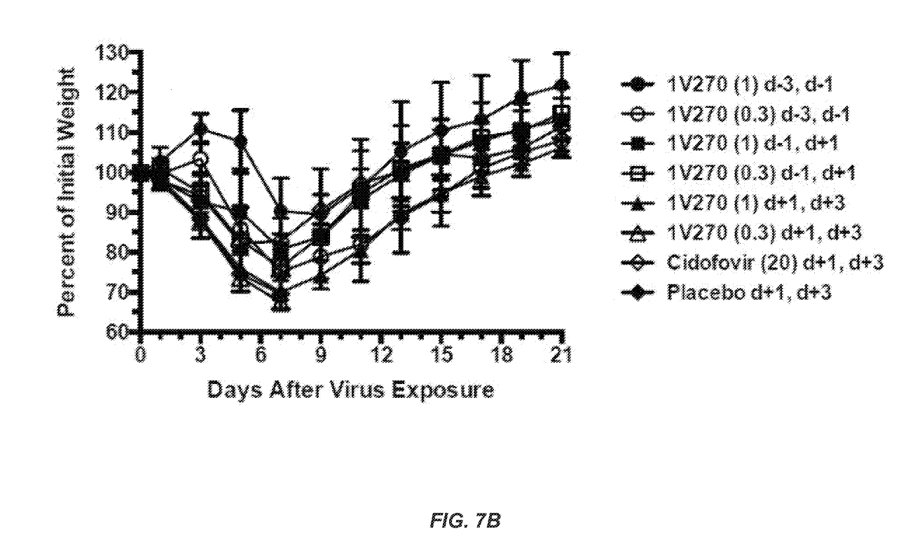

The results of treatment of a lethal vaccinia (IHD strain) infection on mortality of mice are reported in Table 2. Complete protection was afforded by treatment with 1V270 (1 nmol) initiated at day −3, and 90% protection was afforded by the same dose initiated at day −1. When initiated after infection (day +1), then only 30% protection was achieved by this dose. Using the 0.3 nmol dose initiated on days −3, −1, and +1, protection of 50, 80, and 0% was achieved. Thus, the effect of 1V270 was more prophylactic in nature. Survival curves for these results are presented in FIG. 7A. Statistical analysis of survival data takes into account both the numbers of survivors and the delay in time to death, with delay in mortality (if any) being heavily weighted. Since the times to death of mice treated with 1V270 at 1 nmol on days +1 and +3 was similar to that of the placebo group, the difference from placebo was not statistically significant, even though three mice survived as a result of the treatment. Note that the survivors/total differences for the same groups were statistically significant (see Table 2).

TABLE 2

| Compound | | Treatment | Treatment | Survivors/ | Mean Day of |
|---|---|---|---|---|---|
| (Dose)^{a} | | Route | Days^{b} | Total | Death^{c} ± SD |
| 1V270 | (1) | i.n. | −3 and −1 | 10/10*** | — |
| 1V270 | (0.3) | i.n. | −3 and −1 | 5/10** | 8.4 ± 0.5** |
| 1V270 | (1) | i.n. | −1 and +1 | 9/10*** | 9.0 |
| 1V270 | (0.3) | i.n. | −1 and +1 | 8/10*** | 8.0 |
| 1V270 | (1) | i.n. | +1 and +3 | 3/10* | 7.3 ± 0.8 |
| 1V270 | (0.3) | i.n. | +1 and +3 | 0/10 | 7.2 ± 0.6 |
| Cidofovir | (20) | i.p. | +1 and +3 | 10/10 | — |

| | | | | |
|---|---|---|---|---|
| Placebo | i.n. | +1 and +3 | 0/20 | 7.2 ± 0.9 |

Intranasal treatments with 1V270 were given once a day on the indicated days pre-and post-virus exposure. Intraperitoneal treatments with cidofovir were administered once a day on days +1 and +3 after virus challenge
^{a}The 1V270 dose was in nmol/mouse/day. The cidofovir dose was in mg/kg/day.
^{b}Single treatments were administered on the indicated days relative to virus challenge.
^{c}Of mice that died during the 21-day observation period.
*P < 0.05,
**P < 0.01,
***P < 0.001, compared to placebo.

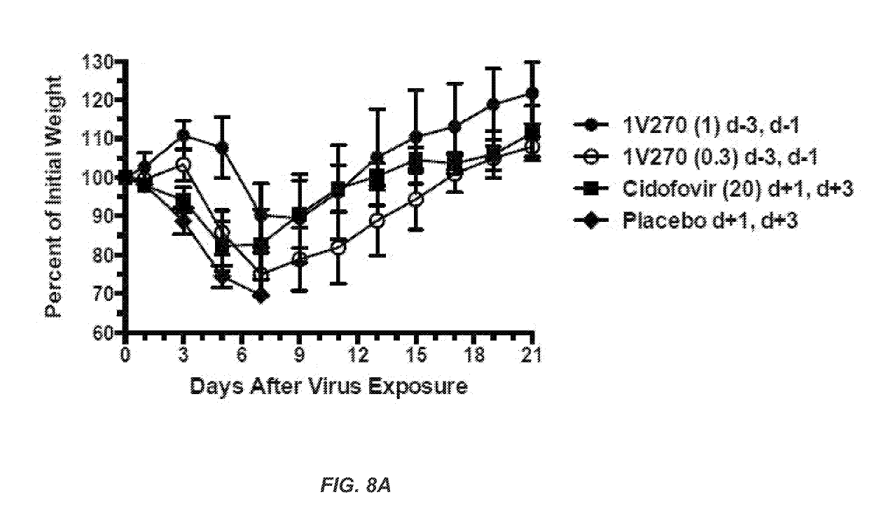

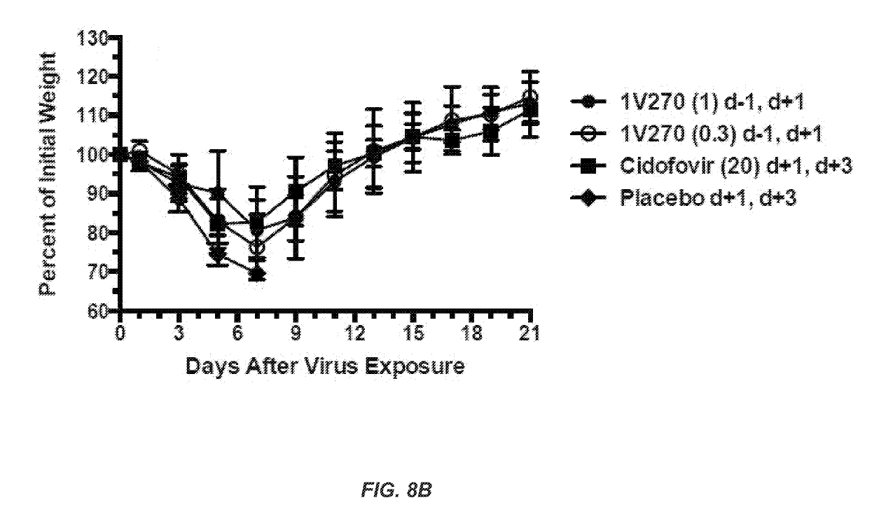

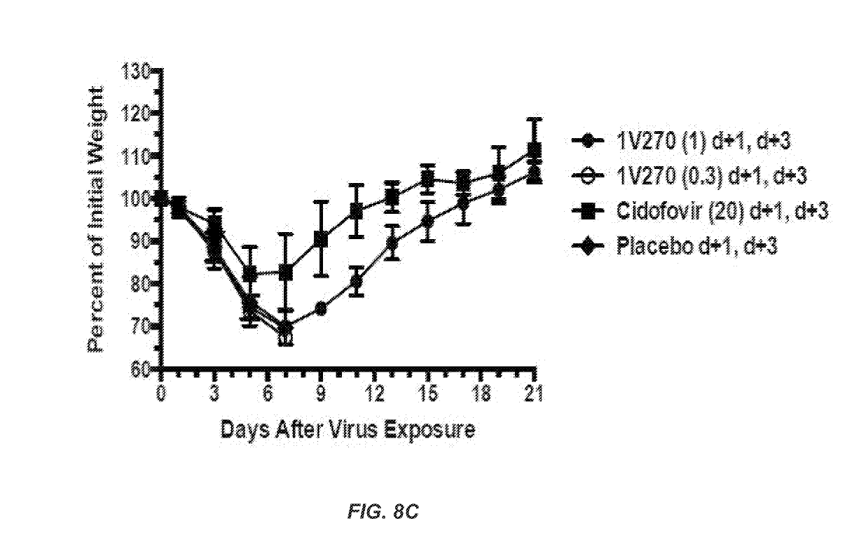

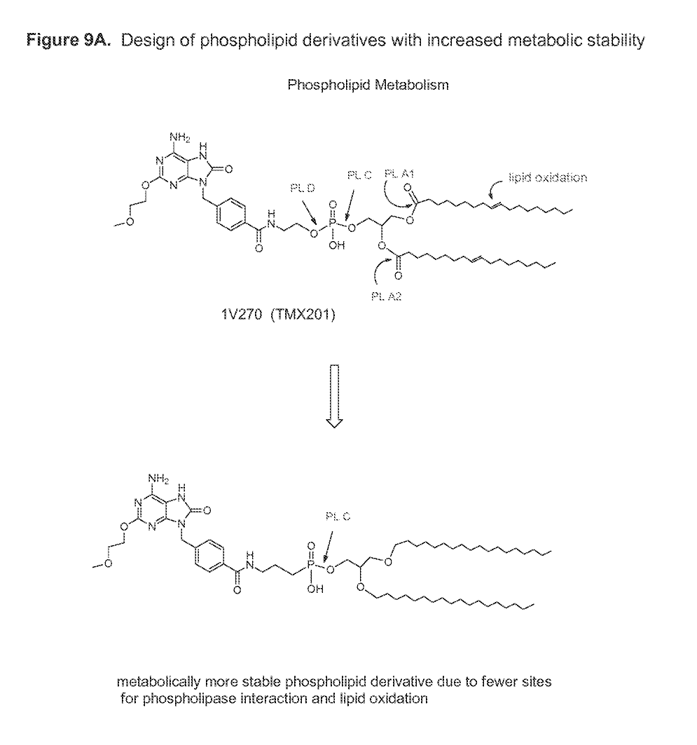

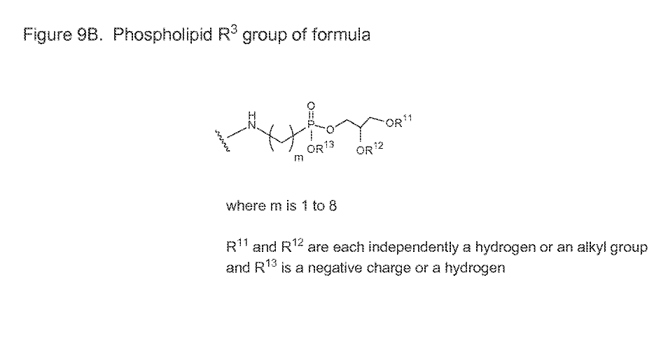

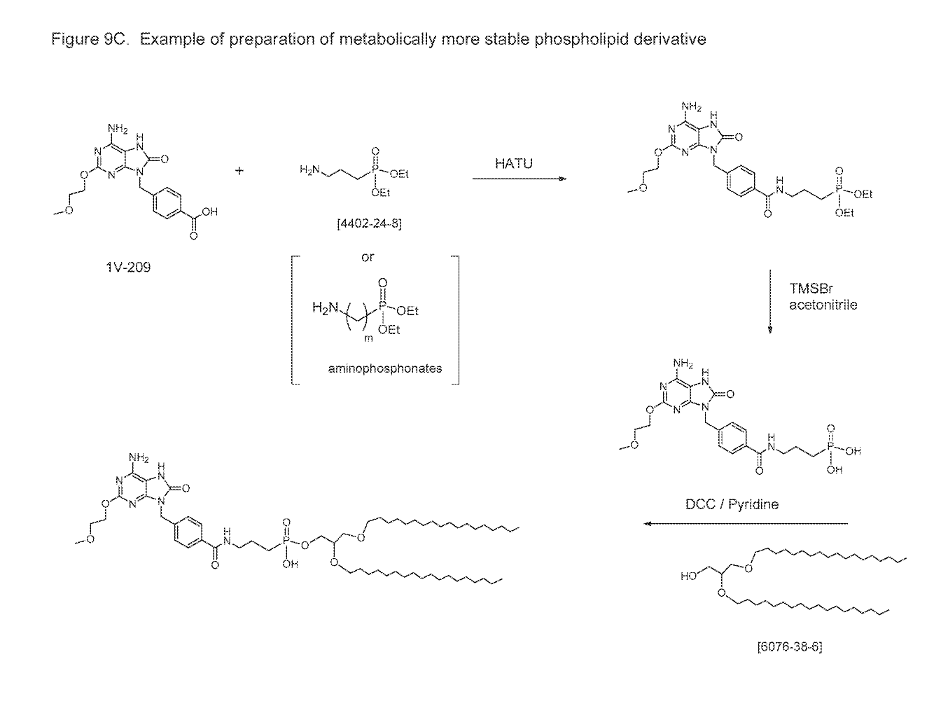

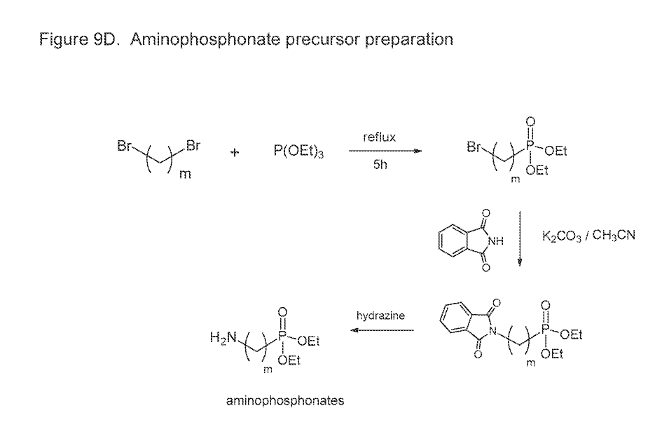

Body weight changes during the infection are shown in FIG. 7B. The body weights in the 1V270 groups that started treatment on day −3 increased over the first three days before declining. This was a better result than that achieved by cidofovir therapy. Since the results were difficult to see due to the many lines of data, they were plotted in smaller groups in FIGS. 8A-C. FIG. 8A provides a clearer picture of the data just described for 1V270 treatment starting on day −3. The best effect in preserving body weight was provided by 1V270 treatments at 1 nmol initiated at day −3, followed by cidofovir, and then by 1V270 at 0.3 nmol. In FIG. 8B, the effects of treatment with 1V270 at 0.3 and 1 nmol for treatments initiated at −1 day were similar to those of cidofovir. In FIG. 8C, it is evident that cidofovir treatments initiated at +1 day were superior to treatments with 1V270 that were started on the same day.

The results indicate that 1V270 exhibits anti-vaccinia virus activity in a mouse model of infection when administered prophylactically. Minimal benefit was achieved by therapeutic administration of the compound. Nevertheless, these results are impressive because efficacy of this immunomodulatory agent alone was able to prevent mortality body weight loss to a high degree.

### CONCLUSIONS

Mice infected with vaccinia (IHD strain) virus were treated prophylactically with 1V270 on days -3 and −1, or days −1 and +1 relative to virus challenge. Other mice were treated therapeutically on days +1 and +3 with 1V270. 1V270 at 1 nmol/mouse/day protected 90-100% mice from death when given prophylactically, but was only 30% protective when treatment was initiated at +1 day. The prophylactic 0.3 nmol/mouse/day dose was 50 to 80% protective when given prophylactically, but was inactive when administered therapeutically. Cidofovir was 100% protective at 20 mg/kg/day, given on days +1 and +3. Prophylactic doses of 1V270 prevented body weight loss in a manner similar to that of cidofovir.

### REFERENCE

1. Smee, D. F., M.-H. Wong, K. W. Bailey, J. R. Beadle, K. Y. Hostetler, and R. W. Sidwell. 2004. Effects of four antiviral substances on lethal vaccinia virus (IHD strain) respiratory infections in mice. Int. J. Antimicrob. Agents 23:430-437.

### Example 4

The optimum dose for 1V270 as treatment of mice for a sub-lethal influenza A/CA/04/2009 (pandemic H1N1) virus infection can be evaluated as follows.

### Study Design (see Table 3)

Virus: Influenza A/CA/04/2009 (pandemic H1N1) virus administered intranasally to 18-20 g BALB/c mice.
Compounds: TLR7 agonist, 1V270.
Number of animals: 10 per treatment group, 10 placebos, 3 normal controls.
Treatment route: intranasal for 1V270, p.o. for oseltamivir.
Start of treatment: 24 h pre-infection, 12, 24, and 48 hour post-infection for 1V270 and oseltamivir.
Treatment times: once for 1V270 and b.i.d.×five days for oseltamivir, oseltamivir treated group will also receive PSS by the intranasal route to match the administration of 1V270.
Parameters for measurement:
   Percent survivors,
   Mean day of death determination,
   Body weight change measured every other day during the infection, and
   Plethysmography to evaluate lung function every other day from day 1-15.

TABLE 3
Experimental design

| No./ | Group | Infected Y | | | | Observations/ |
|---|---|---|---|---|---|---|
| Cage | No. | or N | Compound | Dosage | Treatment Schedule | Testing |
| 10 | 1 | Yes | PSS* | — | 1x i.n., 24 h pre-virus | Weight loss and |
| 10 | 3 | Yes | 1V270 | 200 nM | | mortality through day |
| 10 | 5 | Yes | PSS | — | 1x i.n., 12 h post-virus | 21 |
| 10 | 7 | Yes | 1V270 | 200 nM | | Plethysmography every |
| 10 | 9 | Yes | Oseltamivir | 10 mg/kg/day | b.i.d. p.o. x 5 d beg 12 h post- | other day from day 1-15 |
| | | | | | virus, plus PSS 1x i.n., 12 h | |
| | | | | | post-infection | |
| 10 | 11 | Yes | PSS | — | 1x i.n., 24 h post-virus | |
| 10 | 13 | Yes | 1V270 | 200 nM | | |
| 10 | 15 | Yes | Oseltamivir | 10 mg/kg/day | b.i.d. p.o. x 5 d beg 24 h post- | |
| | | | | | virus plus PSS 1x i.n., 24 h | |
| | | | | | post-infection | |
| 10 | 11 | Yes | PSS | — | 1x i.n., 48 h post-virus | |
| 10 | 13 | Yes | 1V270 | 200 nM | | |
| 10 | 15 | Yes | Oseltamivir | 10 mg/kg/day | b.i.d. p.o. x 5 d beg 24 h post- | |
| | | | | | virus plus PSS 1x i.n., 48 h | |
| | | | | | post-infection | |

| | | | |
|---|---|---|---|
| 5 | 2 | No | Mice observed for normal weight gain |

*PSS—physiological sterile saline

### Examples of Embodiments

Listed hereafter are non-limiting examples of certain embodiments of the technology.
A1. A method to enhance an innate immune response in a mammal, comprising administering to the mammal an effective amount of a compound of Formula (I):

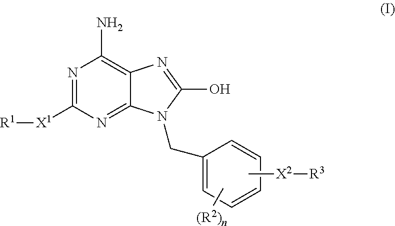

wherein X¹ is —O—, —S—, or —NR^{c}—;

R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;

R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)-(C₁-C₆)alkyl (alkanoyl), substituted —C(O)-(C₁-C₆)alkyl, —C(O)-(C₆-C₁₀)aryl (aroyl), substituted —C(O)-(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

n is 0, 1, 2, 3 or 4;

X² is a bond or a linking group; and

R³ is a phospholipid comprising one or two carboxylic esters;

or a tautomer thereof;
or a pharmaceutically acceptable salt or solvate thereof.
A2. The method of embodiment A1, wherein R³ comprises a group of formula

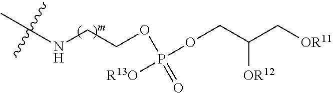

wherein R¹¹ and R¹² are each independently a hydrogen or an acyl group, R¹³ is a negative charge or a hydrogen, and m is 1 to 8, wherein a wavy line indicates a position of bonding, wherein an absolute configuration at the carbon atom bearing OR¹² is R, S, or any mixture thereof.
A3. The method of embodiment A2, wherein m is 1.
A4. The method of embodiment A2 or A3, wherein R¹¹ and R¹² independently are —C(O)-(C₈-C₂₄ alkyl).
A5. The method of any one of embodiments A2 to A4, wherein R¹¹ and R¹² are each oleoyl groups.
A6. The method of embodiment A1, wherein the phospholipid of R³ comprises two carboxylic esters and each carboxylic ester includes one, two, three or four sites of unsaturation, epoxidation, hydroxylation, or a combination thereof.
A7. The method of embodiment A6, wherein each carboxylic ester of the phospholipid is a C18 carboxylic ester with a site of unsaturation at C9-C10.
A8. The method of any one of embodiments A1 to A7, wherein X² is a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups.
A9. The method of embodiment A1, wherein R³ is 1,2-dioleoyl-sn-glycero-3-phosphoethanolamine and X² is C(O).
A10. The method of any one of embodiments A1 to A9, wherein X¹ is oxygen.
A11. The method of any one of embodiments A1 to A10, wherein R¹ is hydrogen, methyl, ethyl, propyl, butyl, hydroxyC₁₋₄alkylene, or C₁₋₄alkoxyC₁₋₄alkylene.
A12. The method of embodiment A1, wherein X¹ is 0, R¹ is C₁₋₄alkoxy-ethyl, n is 0, X² is carbonyl, and R³ is 1,2-dioleoylphosphatidyl ethanolamine (DOPE).
A13. The method of any one of embodiments A1 to A12, wherein administration of the compound is to the respiratory system.
A14. The method of embodiment A13, wherein the administration is by a pulmonary route.
A15. The method of embodiment A13, wherein the administration is by an intranasal route.
A16. The method of any one of embodiments A1 to A15, wherein the innate immune response is effective to prevent or inhibit infection by an infectious agent.
A17. The method of embodiment A16, wherein the compound is administered to the mammal prior to exposure to the infectious agent.
A17.1. The method of embodiment A17, wherein the compound is administered intranasally.
A17.2. The method of embodiment A17, wherein the compound is administered to the mammal 1 to 3 days prior to exposure to the infectious agent.
A18. The method of embodiment A17, wherein the compound is further administered to the mammal after exposure to the infectious agent.
A19. The method of any one of embodiments A1 to A15, wherein the innate immune response is effective to treat infection by an infectious agent.
A20. The method of embodiment A19, wherein the compound is administered to the mammal after exposure to the infectious agent.
A20.1. The method of embodiment A20, wherein the compound is administered intranasally.
A20.2. The method of embodiment A20, wherein the compound is administered to the mammal immediately after exposure to the infectious agent.
A20.3. The method of embodiment A20, wherein the compound is administered to the mammal within one day after exposure to the infectious agent.
A21. The method of any one of embodiments A16-A18, wherein the mammal is exposed to a lethal dose of the infectious agent.
A22. The method of any one of embodiments A16-A20, wherein the mammal is exposed to a sub-lethal dose of the infectious agent.
A23. The method of any one of embodiments A13 to A22, wherein the innate immune response is localized to nasal or respiratory tissues.
A24. The method of any one of embodiments A13 to A22, wherein the innate immune response is not localized to nasal or respiratory tissues.
A25. The method of any one of embodiments A16 to A23, wherein the infection is by inhalation.
A26. The method of any one of embodiments A16 to A24, wherein the infection is by a route other than inhalation.
A27 The method of any one of embodiments A16 to A22, A24 and A26, wherein the infectious agent does not utilize the respiratory system.
A28. The method of any one of embodiments A16 to A27, wherein the infectious agent is a bacteria.
A29. The method of embodiment A28, wherein the bacteria is B. anthracis.
A30. The method of any one of embodiments A16 to A27, wherein the infectious agent is a virus.
A31. The method of embodiment A30, wherein the virus is an influenza virus.
A32. The method of embodiment A30, wherein the virus is an encephalitis virus.
A33. The method of embodiment A30, wherein the virus is a vaccinia virus.
A34. The method of embodiment A30, wherein the virus is West Nile Virus.
A35. The method of any one of embodiments A16 to A27, wherein the infectious agent is a fungus.
A36. The method of any one of embodiments A16 to A35, wherein administration of the compound does not induce detectable off target toxic effects.
A37. The method of any one of embodiments A1 to A36, wherein the mammal is a human.
A38. The method of embodiment A37, wherein the human is immunocompromised.
A39. The method of embodiment A37, wherein the human is elderly or at least 65 years old.
A40. The method of embodiment A37, wherein the human is a young child or less than 5 years of age.
A.41. The method of embodiment A37, wherein the human is pregnant.
A.42. The method of embodiment A37, wherein the human is new born.
B1. A method to enhance an innate immune response in a mammal, comprising administering to the mammal an effective amount of a compound of Formula (I):

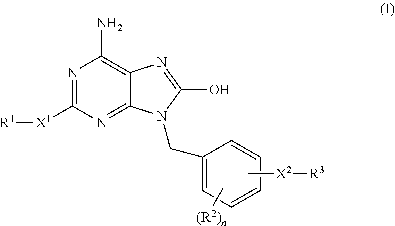

wherein X¹ is —O—, —S—, or —NR^{c}—;

R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;

R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)-(C₁-C₆)alkyl (alkanoyl), substituted —C(O)-(C₁-C₆)alkyl, —C(O)-(C₆-C₁₀)aryl (aroyl), substituted —C(O)-(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

n is 0, 1, 2, 3 or 4;

X² is a bond or a linking group; and

R³ is a phospholipid or analog thereof comprising one or two alkyl ethers or carboxylic esters bonded to the glyceryl moiety thereof;

or a tautomer thereof;
or a pharmaceutically acceptable salt or solvate thereof.
B2. The method of embodiment A1, wherein R³ comprises a group of formula

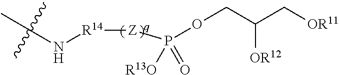

wherein R¹¹ and R¹² are each independently a hydrogen, a C₈-C₂₅ alkyl group or a C₈-C₂₅ acyl group, provided that at least one of R¹¹ and R¹² is an alkyl or an acyl group; R¹³ is a negative charge or a hydrogen, and R¹⁴ is a C₁-C₈ n-alkyl or branched alkyl group which can be substituted or unsubstituted, wherein optionally one of the carbon atoms of the alkyl group is replaced by NH, S, or O; Z is O, S, or NH, and q is 0 or 1;
wherein a wavy line indicates a position of bonding, wherein an absolute configuration at the carbon atom bearing OR¹² is R, S, or any mixture thereof.
B3. The method of embodiment B1or B2, wherein R¹⁴ is C₂ alkyl.
B4. The method of any one of embodiments B1 to B3, wherein R¹¹ and R¹² independently are —C(O)-(C₈-C₂₄ alkyl).
B5. The method of any one of embodiments B1 to B3, wherein R¹¹ and R¹² independently are C₈-C₂₅ alkyl.
B6. The method of embodiment B4, wherein R¹¹ and R¹² are each oleoyl groups.
B7. The method of any one of embodiments B1 to B5, wherein the phospholipid or analog thereof of R³ comprises two carboxylic esters and each carboxylic ester includes one, two, three or four sites of unsaturation, epoxidation, hydroxylation, or a combination thereof.
B8. The method of embodiment B7, wherein each carboxylic ester of the phospholipid or analog thereof is a C₁₈ carboxylic ester with a site of unsaturation at C9-C10.
B9. The method of embodiment B5, wherein one or both of R¹¹ and R¹² is C₁₆, C₁₇, C₁₈ or C₁₉ alkyl.
B10. The method of any one of embodiments B1 to B9, wherein X² is a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups.
B11. The method of any one of embodiments B1 to B9 wherein X² is a carbonyl group.
B12. The method of embodiment B11 wherein the compound of formula (I) is

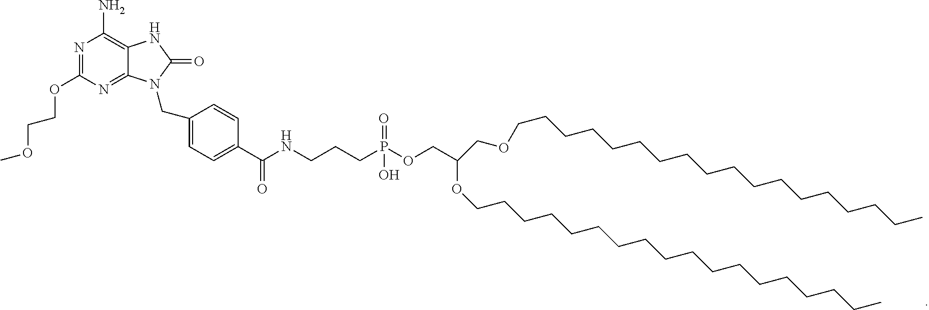 [SMILES: CCCCCCCCCCCCCCCCCCOCC(COP(=O)(O)CCCNC(=O)c1ccc(Cn2c(=O)[nH]c3c(N)nc(OCCOC)nc32)cc1)OCCCCCCCCCCCCCCCCCC]

B13. The method of any one of embodiments B1 to B12, wherein X¹ is oxygen.
B14. The method of any one of embodiments B1 to B13, wherein R¹ is hydrogen, methyl, ethyl, propyl, butyl, hydroxyC₁₋₄alkylene, or C₁₋₄alkoxyC₁₋₄alkylene.
B15. The method of any one of embodiments B1 to B14, wherein administration of the compound is to the respiratory system.
B16. The method of embodiment B15, wherein the administration is by a pulmonary route.
B17. The method of embodiment B15, wherein the administration is by an intranasal route.
B18. The method of any one of embodiments B1 to B17, wherein the innate immune response is effective to prevent or inhibit infection by an infectious agent.
B19. The method of embodiment B18, wherein the compound is administered to the mammal prior to exposure to the infectious agent.
B19.1. The method of embodiment B19, wherein the compound is administered intranasally.
B19.2. The method of embodiment B19, wherein the compound is administered to the mammal 1 to 3 days prior to exposure to the infectious agent.
B20. The method of embodiment B19, wherein the compound is further administered to the mammal after exposure to the infectious agent.
B21. The method of any one of embodiments B1 to B17, wherein the innate immune response is effective to treat infection by an infectious agent.
B22. The method of embodiment B21, wherein the compound is administered to the mammal after exposure to the infectious agent.
B22.1. The method of embodiment B22, wherein the compound is administered intranasally.
B22.2. The method of embodiment B22, wherein the compound is administered to the mammal immediately after exposure to the infectious agent.
B22.3. The method of embodiment B22, wherein the compound is administered to the mammal within one day after exposure to the infectious agent.
B23. The method of any one of embodiments B18 to B20, wherein the mammal is exposed to a lethal dose of the infectious agent.
B24. The method of any one of embodiments B18 to B22, wherein the mammal is exposed to a sub-lethal dose of the infectious agent.
B25. The method of any one of embodiments B15 to B24, wherein the innate immune response is localized to nasal or respiratory tissues.
B26. The method of any one of embodiments B15 to B24, wherein the innate immune response is not localized to nasal or respiratory tissues.
B27. The method of any one of embodiments B18 to B25, wherein the infection is by inhalation.
B28. The method of any one of embodiments B18 to B26, wherein the infection is by a route other than inhalation.
B29. The method of any one of embodiments B18 to B24, B26 and B28, wherein the infectious agent does not utilize the respiratory system.
B30. The method of any one of embodiments B18 to B29, wherein the infectious agent is a bacteria.
B31. The method of embodiment B30, wherein the bacteria is B. anthracis.
B32. The method of any one of embodiments B18 to B29, wherein the infectious agent is a virus.
B33. The method of embodiment B32, wherein the virus is an influenza virus.
B34. The method of embodiment B32, wherein the virus is an encephalitis virus.
B35. The method of embodiment B32, wherein the virus is a vaccinia virus.
B36. The method of embodiment B32, wherein the virus is West Nile Virus.
B37. The method of any one of embodiments B18 to B29, wherein the infectious agent is a fungus.
B38. The method of any one of embodiments B18 to B37, wherein administration of the compound does not induce detectable off target toxic effects.
B39. The method of any one of embodiments B1 to B38, wherein the mammal is a human.
B40. The method of embodiment B39, wherein the human is immunocompromised.
B41. The method of embodiment B39, wherein the human is elderly or at least 65 years old.
B42. The method of embodiment B39, wherein the human is a young child or less than 5 years of age.
B43. The method of embodiment B39, wherein the human is pregnant.
B44. The method of embodiment B39, wherein the human is new born.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1-61. (canceled)

62. A compound of Formula (I): 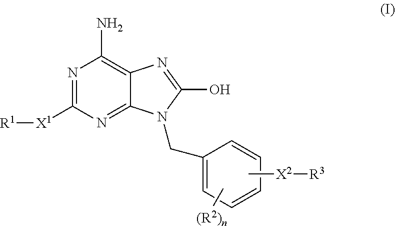
wherein X¹ is —O—, —S—, or —NR^{c}—;
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, or substituted C₅₋₉heterocyclic;
R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)-(C₁-C₆)alkyl (alkanoyl), substituted —C(O)-(C₁-C₆)alkyl, —C(O)-(C₆-C₁₀)aryl (aroyl), substituted —C(O)-(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;
n is 0, 1, 2, 3 or 4;
X² is a bond or a linking group; and
R³ is a phospholipid or analog thereof comprising at least one alkyl ether bonded to the glyceryl moiety thereof;
or a tautomer thereof;
or a pharmaceutically acceptable salt or solvate thereof.

63. The compound of claim 62, wherein R³ comprises a group of formula 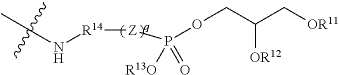
wherein R¹¹ and R¹² are each independently a hydrogen or a C₈-C25 alkyl group; R¹³ is a negative charge or a hydrogen, and R¹⁴ is a C₁-C₈ n-alkyl or branched alkyl group which can be substituted or unsubstituted, wherein optionally one of the carbon atoms of the alkyl group is replaced by NH, S, or O; Z is S or NH, and q is 0 or 1;
wherein a wavy line indicates a position of bonding, wherein an absolute configuration at the carbon atom bearing OR² is R, S, or any mixture thereof.

64. The compound of claim 63, wherein R¹⁴ is C₂ alkyl.

65. The compound of claim 63, wherein R¹¹ and R¹² independently are —C(O)—(C₈-C₂₄ alkyl).

66. The compound of claim 63, wherein and R¹² independently are C₈-C₂₅ alkyl.

67. The compound of claim 62, wherein R³ is a phospholipid or analog thereof comprising two alkyl ethers.

68. The compound of claim 63, wherein one or both of R¹¹ and R¹² are C₁₆, C₁₇, C₁₈ or C₁₉ alkyl.

69. The compound of claim 63, wherein q is 0.

70. The compound of claim 63, wherein R¹⁴ is a C₁-C₈ n-alkyl group which can be substituted or un substituted.

71. The compound of claim 63, wherein one of R¹¹ or R¹² s a carboxylic ester bonded to the glyceryl moiety thereof.

72. The compound of claim 71, wherein the carboxylic ester is a C₈-C₂₅ acyl group.

73. The compound of claim 62, wherein X² is a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups.

74. The compound of claim 62, wherein X² is a carbonyl group.

75. The compound of claim 62, wherein X¹ is oxygen.

76. The compound of claim 62, wherein R¹ is hydrogen, methyl, ethyl, propyl, butyl, hydroxyC₁₋₄alkylene, or C₁₋₄alkoxyC₁₋₄alkylene.

77. The compound of claim 62, wherein R¹ is C₁₋₄alkoxy-ethyl.

78. The compound of claim 62, wherein the compound of formula (I) is

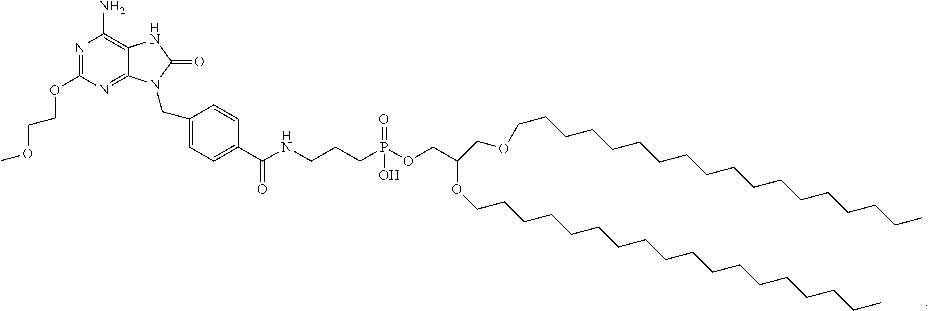 [SMILES: CCCCCCCCCCCCCCCCCCOCC(COP(=O)(O)CCCNC(=O)c1ccc(Cn2c(=O)[nH]c3c(N)nc(OCCOC)nc32)cc1)OCCCCCCCCCCCCCCCCCC]

79. A pharmaceutical composition comprising the compound of claim 62.

80. A method to enhance an innate immune response in a mammal, comprising administering to the mammal an effective amount of the compound of claim 62.

81. The method of claim 80, wherein administration of the compound is to the respiratory system.
